(19) United States
(12) Patent Application Publication
(10) Pub. No.: US 2016/0151024 A1
(43) Pub. Date: Jun. 2, 2016

(54) RADIATION TOMOGRAPHIC APPARATUS

(71) Applicant: HITACHI MEDICAL CORPORATION, Tokyo (JP)

(72) Inventors: Taiga GOTO, Tokyo (JP); Hisashi TAKAHASHI, Tokyo (JP)

(21) Appl. No.: 14/906,432

(22) PCT Filed: Aug. 6, 2014
(86) PCT No.: PCT/JP2014/070674

§ 371 Date: Jan. 20, 2016

(30) Foreign Application Priority Data
Aug. 14, 2013 (JP) 2013-168637

(51) Int. Cl. A61B 6/04; A61B 6/00; A61B 6/03

(52) U.S. Cl. CPC A61B 6/0457; A61B 6/032; A61B 6/0407; A61B 6/547; A61B 6/5205; A61B 6/461

(57) ABSTRACT

In order to provide a CT image with high-spatial resolution and a CPR image without deterioration due to interpolation by scanning while always moving a site of interest of an object to the scanning center, a bed on which the object is placed is moved in a direction orthogonal to the body-axis direction of the object or a scanner angle is changed when scanning the object while moving it in a direction crossing the rotation direction i.e., the body-axis direction of the object, during the rotation of a rotary disk where a radiation source and a radiation detector are disposed oppositely. Image reconstruction is performed using bed movement information (movement information in a direction orthogonal to the body-axis direction) or scanner angle information during scanning.

### TECHNICAL FIELD

The present invention relates to a radiation tomographic apparatus such as an X-ray CT apparatus, and in particular to an image reconstruction method in a radiation tomographic apparatus equipped with a mechanism for moving a bed in a direction orthogonal to the body-axis direction of an object during scanning. Also, the present invention relates to a technique for scanning a site of interest located along a direction approximately parallel to the body-axis direction of the object with high spatial resolution.

### BACKGROUND ART

An X-ray CT apparatus is an apparatus that places an object in the central opening of a rotary disk disposed oppositely to an X-ray source and an X-ray detector and scans the object to generate a tomographic image by rotating the rotary disk. Such an X-ray CT apparatus acquires a plurality of tomographic images for a predetermined region along the body-axis direction by changing positions of the object and the rotary disk relatively to the body-axis direction when the site of interest is located along a direction approximately parallel to the body-axis direction of the object in order to extract an image of the site of interest from these tomographic images.

Generally, an X-ray CT apparatus is designed so as to increase spatial resolution in the scanning center. In the patent literature 1, it is suggested that a site of interest is moved to the scanning center before scanning.

### CITATION LIST

### Patent Literature

### PTL 1: Japanese Unexamined Patent Application Publication No. 2007-267783

### PTL 2: Japanese Unexamined Patent Application Publication No. 2004-188163

### PTL 3: Japanese Unexamined Patent Application Publication No. 9-19425

### SUMMARY OF INVENTION

### Technical Problem

In the technique described in the above PTL 1, the scanning center is moved for each scanning region before scanning. However, because a site of interest such as a blood vessel does not always run parallel to the body-axis direction, the site of interest cannot be always disposed in the scanning center.

The present invention has a purpose to provide a method for solving the above problem occurring in a radiation tomographic apparatus such as an X-ray CT.

### Solution to Problem

In order to solve the above problem, the radiation tomographic apparatus of the present invention is provided with means for moving a bed where an object is placed in a direction orthogonal to the body-axis direction of the object or changing a scanner angle during scanning and reconstructing an image using movement information of the bed during scanning (movement information in the direction orthogonal to the body axis) or an angle change amount of the scanner in image reconstruction.

That is, the radiation tomographic apparatus of the present invention is provided with a bed that can place an object and move in the body-axis direction of the object, a rotary disk that disposes a radiation source irradiating a radiation and a radiation detector oppositely across the bed and rotates around the bed, an image generation unit that reconstructs a tomographic image of the object based on radiation data detected by the radiation detector while the rotary disk is rotating, a mechanical unit that changes a position of the bed in a direction orthogonal to the body-axis direction and/or an angle to the vertical plane of the rotary disk, a control unit that controls the mechanical unit, and a movement amount setting unit that sets a movement amount of the bed in a direction orthogonal to the body-axis direction and/or an angle change amount of the rotary disk during scanning. The control unit controls the mechanical unit to perform scanning according to the bed movement amount and/or the angle change amount of the rotary disk set by the movement amount setting unit. The image generation unit generates an image using movement information of the bed in a direction orthogonal to the body-axis direction and/or angle information of the rotary disk during scanning.

### Advantageous Effects of Invention

According to the present invention, when a site of interest of an object is a blood vessel, the gastrointestinal tract, the spine, or the like that has a structure extending in a predetermined direction, the site of interest can be always disposed so as to be at an approximately center of scanning. Consequently, an image of high spatial resolution can be acquired for the site of interest with a low exposure.

The other various effects of the present invention will be explained in each embodiment to be described later.

### BRIEF DESCRIPTION OF DRAWINGS

FIG. 1 is an external view showing an example of the X-ray CT apparatus to which the present invention is applied.

FIG. 2 is a block configuration diagram showing an embodiment of the X-ray CT apparatus of the present invention.

FIG. 3 is a diagram showing examples of the scan modes to be adopted by the X-ray CT apparatus of the present invention. (a) shows the normal scan method, (b) shows the helical scan method, and (c) shows the shuttle scan method.

FIG. 4 is a diagram explaining a bed movement direction.

FIG. 5 is a diagram showing an embodiment of the operation procedure of the X-ray CT apparatus of the present invention.

FIG. 6 is a diagram explaining a bed movement curve setting.

FIG. 7 is a diagram explaining a bed movement curve setting.

FIG. 8 is a diagram showing display examples of a display device when the bed movement curve is set. (a) shows a case where a coronal image is displayed, and (b) shows a case where a sagittal image is displayed.

FIG. 9 is a diagram showing the scanning methods by the present invention and the effects of the image reconstruction method. (a) is a diagram showing the projection data by a conventional scanning method and the reconstruction image, (b) is a diagram showing the projection data by scanning while the bed is moving horizontally and vertically and the reconstruction image by a conventional reconstruction method, and (c) is a diagram showing the projection data by scanning while the bed is moving horizontally and the reconstruction image by the reconstruction method of the present invention.

FIG. 10 is a diagram explaining a tilt angle change of the scanner.

FIG. 11 is a diagram showing the operation procedure of the second embodiment.

FIG. 12 is a diagram showing relationships between an object size and an X-ray detector size. (a) shows a case where a detector size is large enough to cover the entire object, and (b) shows a case where a detector size is too small to cover the entire object.

FIG. 13 is a diagram explaining a bed movement scan by the third embodiment.

FIG. 14 is a diagram explaining a bed movement scan by the fourth embodiment. (a) shows a sampling density of a conventional method, and (b) shows a sampling density of the fourth embodiment.

FIG. 15 is a diagram showing a bed movement curve in the fourth embodiment. (a) shows a case where a bed is moved continuously in the horizontal or vertical direction, and (b) shows a case where a bed is moved intermittently in the horizontal or vertical direction.

FIG. 16 is a diagram explaining a bed movement scan by the fourth embodiment. (a) shows image reconstruction (conventional method) in a case where a bed is not moved, and (b) shows image reconstruction (the fourth embodiment) in a case where a bed is moved.

FIG. 17 is a diagram explaining image synthesization in the fifth embodiment. (a) is a diagram showing an image before synthesization, and (b) is a diagram showing an image after synthesization.

FIG. 18 is a diagram explaining CPR image reconstruction in the sixth embodiment. (a) is a diagram showing CPR image generation by a conventional method, and (b) is a diagram showing a concept of the CPR image generation by the sixth embodiment.

FIG. 19 is a diagram showing a display window example in a display device of the X-ray CT apparatus of the seventh embodiment.

FIG. 20 is a diagram showing the flow of the image reconstruction process in the seventh embodiment.

### DESCRIPTION OF EMBODIMENTS

An embodiment in which the radiation tomographic apparatus of the present invention is applied to an X-ray CT apparatus will be described.

The X-ray CT apparatus of the present embodiment is comprised of the bed 20 that can place an object and move in the body-axis direction of the object, a rotary disk (the scanner 10) that disposes the X-ray source 11 irradiating an X-ray and the X-ray detector 12 oppositely across the bed 20 and rotates around the bed, an image generation unit (the reconstruction computing unit 32 and the image processing unit 33) that reconstructs a tomographic image of the object based on X-ray data detected by the X-ray detector 12 while the rotary disk is rotating, a mechanical portion (the mechanical unit 25 and the tilt mechanical unit 151) that change a position of the bed and/or an angle to the vertical plane of the rotary disk, a control portion (the central controller 40) that controls the mechanical portion, and a movement amount setting unit (the bed movement amount setting unit 31) that sets a movement amount in a direction orthogonal to the body-axis direction of the bed and/or an angle change amount of the rotary disk during scanning.

The control unit 40 controls the mechanical portion (25 and 151) to perform scanning according to the movement amount of the bed and/or the angle change amount of the rotary disk set by the movement amount setting unit 31. The image generation unit (32 and 33) generates an image using movement information in a direction orthogonal to the body-axis direction of the bed and/or angle information of the rotary disk during scanning.

The image generation unit generates an image using, for example, a movement amount in a direction orthogonal to the body-axis direction of the bed and/or an angle change amount of the rotary disk set by the movement amount setting unit. Also, in a case where the mechanical portion (25 and 151) is provided with a measurement portion (the bed movement measuring unit 23) that measures a movement amount in a direction orthogonal to the body-axis direction of the bed and/or an angle change amount of the rotary disk during scanning, the image generation unit generates an image using the movement amount of the bed 23 and/or the angle change amount of the rotary disk recorded by the measuring unit.

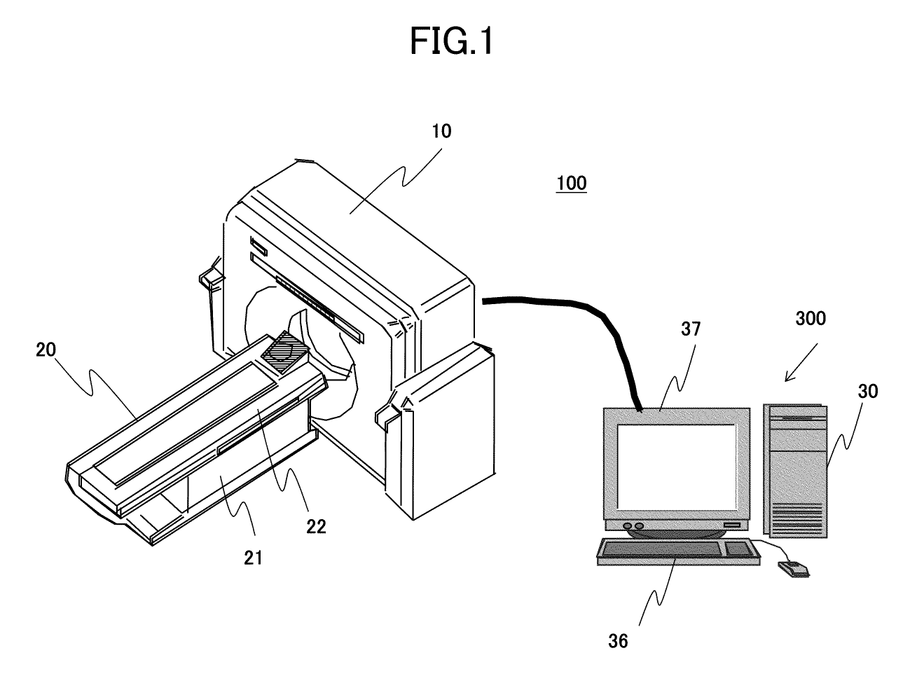

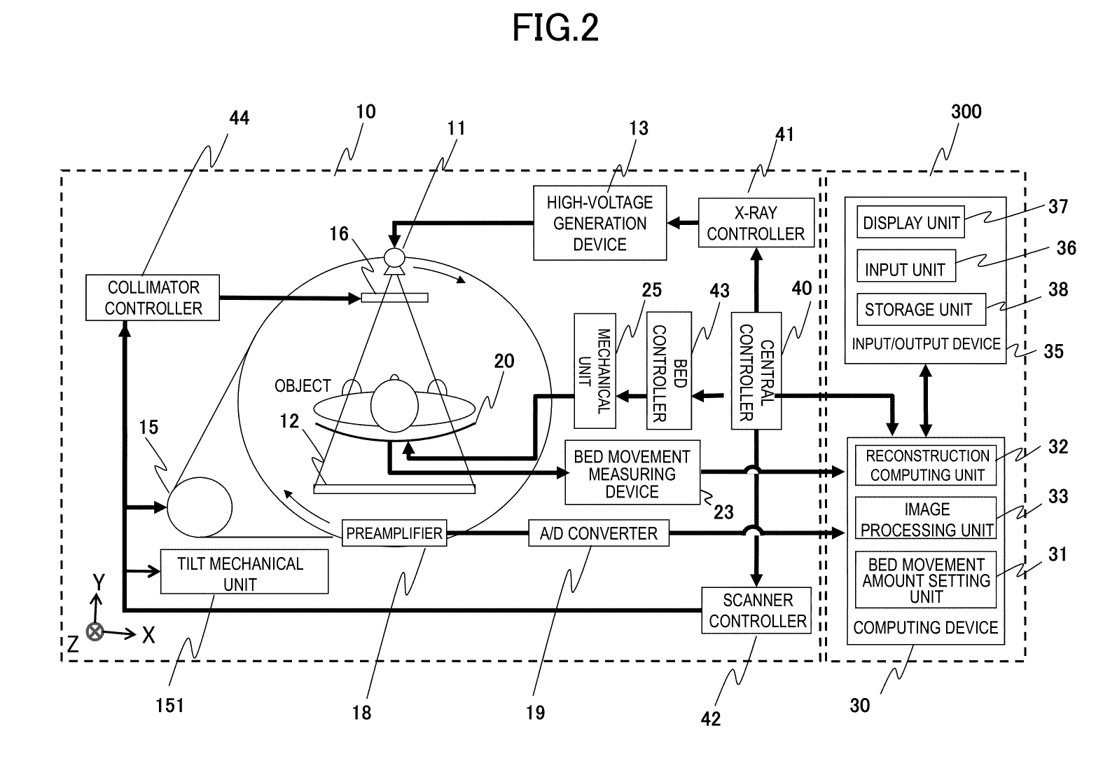

Hereinafter, referring to the diagrams, the X-ray CT apparatus of the present embodiment will be described. FIG. 1 is a diagram showing the external view of the X-ray CT apparatus 100 of the present embodiment, and FIG. 2 is a diagram showing the overall configuration. This X-ray CT apparatus is provided with the scanner 10 that scans an object, the bed 20 on which the object is placed, and the operation unit 300. The operation unit 300 is provided with the computing device that performs computation such as image reconstruction and the input/output device 35 that includes the input unit 36, the display unit 37, and the like.

The scanner 10 accommodates the rotary disk (not shown in the diagrams) that oppositely disposes the X-ray generation device 11 and the X-ray detector 12 oppositely in the gantry that has an opening in the center and performs scanning by rotating the rotary disk at a predetermined rotational speed. The scan mode is not limited, but the rotate-rotate method (the third generation) is adopted.

Inside the scanner 10, accommodated are the high-voltage generation device 13 that supplies high voltage to the X-ray generation device 11, the X-ray controller 41 that controls the high-voltage generation device 13 and irradiates an X-ray from the X-ray generation device, the driving device 15 of the rotary disk, the scanner controller 42 that controls the driving device 15, the mechanical unit 25 for moving the bed 20, the bed controller 43 that controls the mechanical unit 25, the collimator controller 44 that controls the collimator 16 provided with the X-ray generation device 11, the central controller 40 that controls the respective controllers 41 to 44, and the like.

The X-ray detector 12 is, for example, a multi-row detector (two-dimensional detector) in which a lot of X-ray detection elements are arranged along the rotational direction of the rotary disk in a detector row and a plurality of the detector rows are arranged in a direction orthogonal to the rotational direction, and each of the detection elements generates an electrical signal corresponding to the detected X-ray amount. The scanner 10 is provided with the preamplifier 18 that amplifies an output of the X-ray detector 12 and the A/D converter 19. A digital signal that is an output of the A/D converter 19 is input to the computing device 30.

The scanner 10 can be provided with the tilt mechanical unit 151 that can change an angle to the upper plane of the bed 20. When a plane vertical to the longitudinal direction of the bed 20 is a reference plane, an angle of the scanner (rotary disk) 10 to the reference plane is referred to as a tilt angle. A tilt angle can be changed in a range where the opening of the scanner 10 does not interfere with the bed 20, and the range is approximately ±30 degrees normally.

The bed 20 is provided with the supporting stand 21 and the top plate 22 installed on the supporting stand 21, and the top plate 22 can be moved in a direction along the body-axis direction of an object (here, the Z direction), which can convey the object placed on the bed 20 to a scanning position in the opening of the scanner 10. The bed 20 is provided with a mechanism that moves the top plate 22 in the Z direction in addition to the mechanical unit 25 that moves in a direction orthogonal to the Z direction such as the up-and-down direction (the vertical direction, the Y direction) and the left-and-right direction (the horizontal direction orthogonal to the body-axis direction, the X direction). The mechanical unit 25 can use publicly known drive mechanisms such as a stepping motor and linear motor. The mechanical unit 25 is controlled by the bed controller 43. The bed controller 43 can change positions in the X and Y directions of the bed 20 by driving the mechanical unit 25 during scanning. The bed movement measuring device 23 is provided in the vicinity of the bed 20 to measure a movement amount in each direction of the bed 20 and output it to the computing device 30 as movement information. Additionally, although it is desirable to provide the bed movement measuring device 23 in the present embodiment, this is not necessarily required. In a case where the bed movement measuring device 23 is provided, the bed position information measured by the bed movement measuring device 23 is sent to the computing device 30 to be used for image generation.

The input/output device of the operation unit 300 is provided with the input unit 36 such as a pointing device including a keyboard and a mouse, the display unit 37, and the storage unit 38 storing parameters, data, computation results, and the like required for computation of the computing device 30. The computing device 30 is operated under the control of the central controller 40 and provided with the reconstruction computing unit 32 that performs image reconstruction computation using a signal sent from the X-ray detector 12 through the A/D converter 19 and the image processing unit 33 that performs correction and the like of the image reconstructed by the reconstruction computing unit 32. The reconstruction computing unit 32 and the image processing unit 33 function as the image generation unit of the present invention. The computing device 30 further has the movement amount setting unit 31 that sets a movement amount of the bed.

Operations of the X-ray CT apparatus will be described briefly.

Scanning conditions (a tube current, a tube voltage, a rotational speed, and a bed movement speed in the body-axis direction), reconstruction conditions (a reconstruction mode, an image FOV, a reconstruction filter, an image slice thickness, and a reconstruction slice position), and the other processing condition (a CPR mode) are input from the input unit 36 of the operation unit 300, a control signal required for scanning is sent to the X-ray controller 41, the bed controller 43, and the scanner controller 42 from the central controller 40 based on the input conditions, and then scanning starts after receiving a scanning start signal. When the scanning starts, the control signal is sent to the high-voltage generation device 13 by the X-ray controller 41, a high voltage is applied to the X-ray generation device 11, and then an X-ray is irradiated to an object from the X-ray generation device 11. Simultaneously, the control signal is sent from the scanner controller 42 to the driving device 15 in order to rotate the X-ray generation device 11, the X-ray detector 12, and the preamplifier 18 around the object. At this time, a bed where the object is placed is moved in the body-axis direction (Z direction) of the object by the bed controller 43 according to the scan mode.

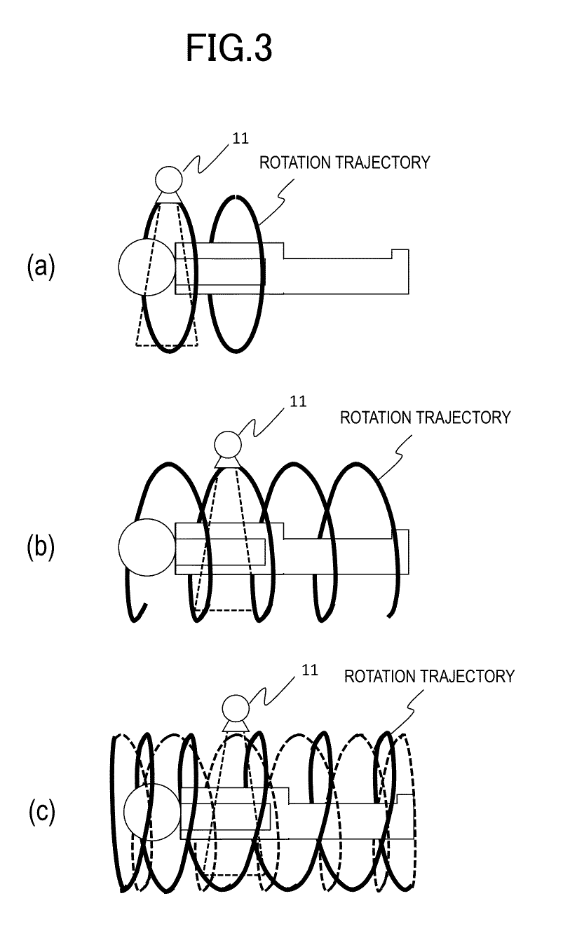

The scan mode includes the mode (a) (here referred to as the normal scan mode) that moves a bed gradually in the body-axis direction for each rotation of the scanner for example, the mode (b) (the helical scan mode) that continuously moves the bed in the body-axis direction, the mode (c) (the shuttle scan mode) that moves the bed forward and backward in the body-axis direction, and the like as shown in FIG. 3. Although not shown in the diagram, there is also a mode (the variable pitch scan) that changes a bed movement speed during scanning while moving the bed in the body-axis direction.

An X-ray irradiated from the X-ray generation device 11 is absorbed (attenuated) by each tissue of an object, transmits through the object, and is detected by the X-ray detector 12 after the irradiation range is limited by the collimator 16.

The X-ray detected by the X-ray detector 12 is converted into a current, amplified by the preamplifier 18, and then input to the computing device 30 as a projection data signal after A/D conversion is performed by the A/D converter. The image reconstruction process is performed for the projection data signal input by the computing device 30 in the reconstruction computing unit 32 in the computing device 30.

The reconstruction image is stored in the storage unit 38 in the input/output device 35 and displayed as a CT image on the display unit 37. Alternatively, reconstruction image is displayed as a CT image on the display unit 37 after being processed in the image processing unit 33.

The X-ray CT apparatus of the present embodiment is characterized by that a bed movement amount is set by the movement amount setting unit 31 in the above scanning before performing scanning while moving the bed 20 in a direction orthogonal to the Z direction (the horizontal direction and/or horizontal direction) based on the set movement amount or performing scanning while changing a tilt angle of a scanner mode.

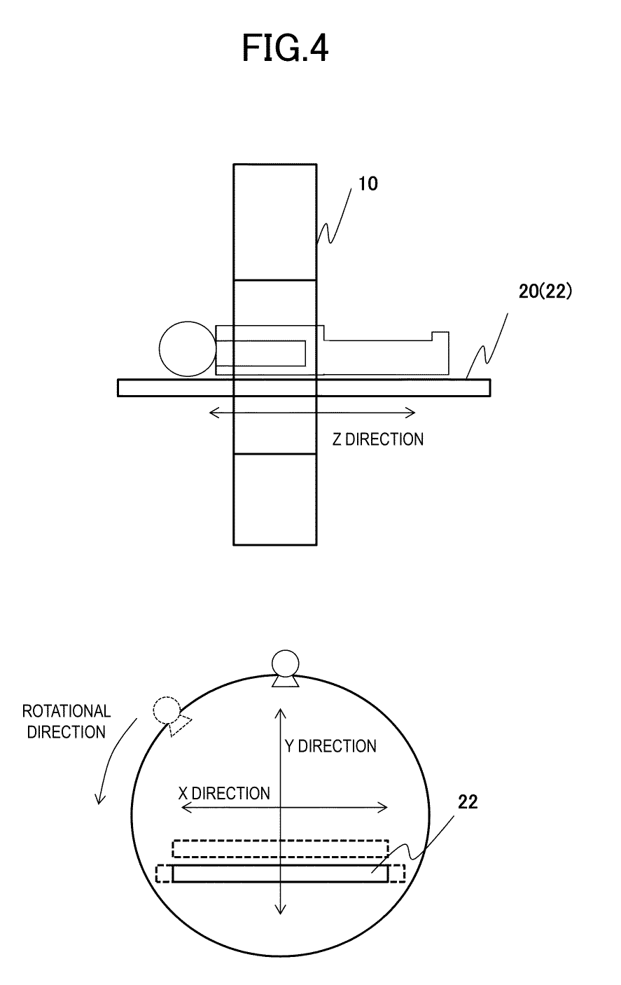

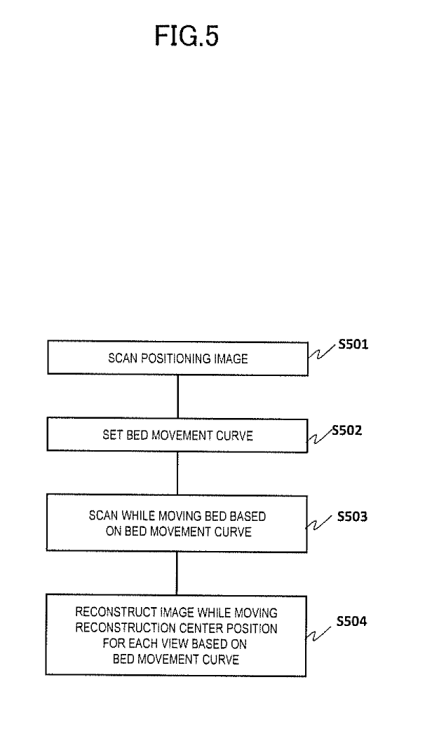

Hereinafter, taking a case of moving a bed as an example, the basic operations of the X-ray CT apparatus of the present embodiment will be described. FIG. 4 is a diagram explaining a bed movement direction, and FIG. 5 is a diagram showing the operation procedure.

As shown in FIG. 4, the top plate portion of the bed 20 on which an object is to be placed is almost parallel to the floor surface, and the longitudinal direction corresponds to the body-axis direction (Z direction) of the object. Although a direction orthogonal to the Z direction is an arbitrary direction in a plane orthogonal to the Z axis, a case where movement is possible in the horizontal direction (X direction) and in the vertical direction (Y direction) of the object will be described here.

First, pre-scanning is performed (S501). Pre-scanning is performed to acquire information about an object required for main scanning to be performed later and acquires image data at a relatively low resolution. Any of the scan modes shown in FIG. 3 is available, and scanning is performed at a relatively low resolution, which increases a movement speed in the body-axis direction, for example, to a rotational speed of the rotary disk in order to acquire volume projection data with a low exposure in a short time. This volume projection data is set as image data by a publicly known image reconstruction method (hereinafter, referred to as pre-scan image data). An X-ray CT apparatus generally acquires a volume image of a low spatial resolution as a positioning image in order to dispose a site of interest of the object at an appropriate position of the scanning system in scanning. This positioning image may be used for pre-scan image data of the present embodiment.

Next, based on the pre-scan image data, a bed movement curve is set (S502). The bed movement curve is a curve in which a bed movement amount (a movement amount in a direction orthogonal to the body-axis direction, for example, the X direction and/or Y direction) was set for a view angle. The specific shape of the bed movement curve is different depending on the scanning purpose and the site of interest of an object, and there are, for example, a curve showing a bed movement amount required for positioning the site of interest of the object in the scanning center (the rotation center of the rotary disk), a curve changing to a linear shape within a predetermined range, and the like.

The bed movement curve will be described in detail later, but an examiner can set the bed movement curve interactively on the display window by displaying a pre-scan image on a display device, and the image processing unit 33 can also generate the bed movement curve automatically by connecting pixels in a site of interest for each slice based on pre-scan image data.

Main scanning starts after the bed movement curve is set (S503). In the main scanning, projection data is acquired over a predetermined range while moving a bed based on the bed movement curve. Any of the scan modes shown in FIG. 3 is available also here, and scanning is similar to the normal one except requiring movement of the bed 20 in the X/Y direction with rotation of the rotary disk.

An image is reconstructed using the projection data after scanning (S504). Although publicly known reconstruction algorithms such as a filter correction reverse projection method, the expansion methods (for example, the methods described in PTL 2 and PTL 3), and a successive approximation reconstruction method can be used for the image reconstruction, a position coordinate of the scanning center is converted using position information for each view in the reconstruction arithmetic expression. The position information can be calculated from the bed movement curve set in Step S502. Also, in a case where the X-ray CT apparatus is provided with the bed movement measuring device 23 of the bed 20, the bed position information measured by the bed movement measuring device 23 can be used. Because the position information uses an actual position, more accurate image reconstruction can be performed than a case of using the set bed movement curve.

Based on the above-described scheme of the apparatus and operations of the present embodiment, the respective embodiments in which the movement form and the use form of the position information of a bed or a scanner are different will be described in detail.

### First Embodiment

The X-ray CT apparatus of the present embodiment is characterized by that a bed position is controlled so that the scanning center moves along the center line of a site of interest during scanning. That is, the movement amount setting unit setting a bed movement amount sets a bed movement amount so as to locate a site of interest of an object in the rotation center of the rotary disk (the scanning center).

Hereinafter, referring to the scanning procedure shown in FIG. 5, operations of the X-ray CT apparatus in the present embodiment will be described.

Based on the image data acquired by pre-scanning (S501), a bed movement curve is set (S502). Although the bed movement curve can be set automatically, the procedure in a case where an operator specifies the setting interactively will be described hereinafter.

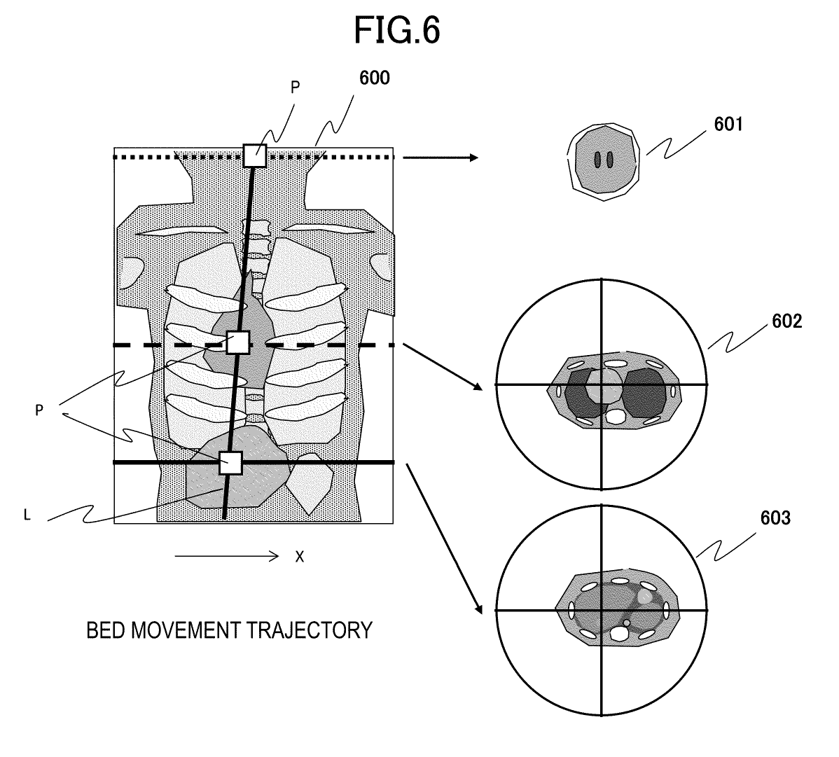

First, as shown in FIG. 6, the pre-scan image (here, a coronal image) 600 is displayed on the display unit 37. The control point P is set in a site of interest to be moved to the scanning center on this image. At the same time, it may be configured so that cross sections setting the control points P are specified on the image and the images 601 to 603 of the cross sections are displayed on the sub-window in order to set the control points P on these tomographic images 601 to 603. The bed movement amount setting unit 31 of the computing device 30 generates the bed movement curve L by connecting these multiple control points P that were specified. The bed movement curve L becomes a movement curve that moves a bed in the left-and right direction of an object (X direction) in a case where a control point is specified and generated on the pre-scan image 600. In a case where control points are specified on the tomographic images 601 to 603, the bed movement curve L becomes a movement curve that includes movement in the up-and-down direction in addition to the left-and right direction. Additionally, the bed movement curve may be formed by connecting a plurality of the control points P linearly or by connecting them with non-linear interpolation using a spline curve or the like.

The bed movement curve is a curve showing a movement amount in the horizontal direction and/or vertical direction to the position in the body-axis direction, and the bed position in the body-axis direction during scanning is a function of the view angle (projection angle of a fan beam) β and can be described in the following formulas (1-1) and (1-2).

[Formula 1]

κₓ=f1(β) (1-1)

κ_{y}=f2(β) (1-2)

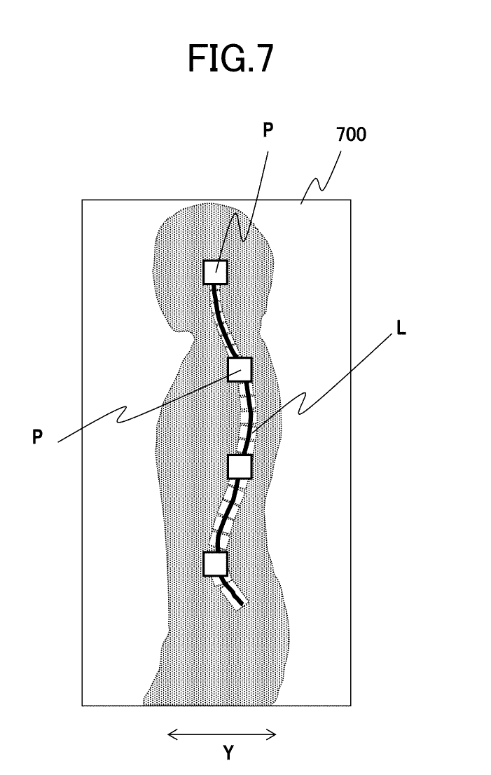

In a case where a site of interest is the spine, a part along the spine, or the like of an object, the pre-scan image may be the image 700 of the sagittal plane shown in FIG. 7. In this case, the control points may be specified on the image 700, or the tomographic images in a plurality of positions different in the body-axis direction may be displayed in order to specify the control points.

Additionally, in case of automatically setting a bed movement curve, features on the image of a site of interest (distribution of pixel values and a feature value) are determined, pixels satisfying features or center pixels of regions satisfying features are connected as the control points, which can form the bed movement curve.

Here, a bed movement range is limited in the vertical and horizontal directions by a bed movement mechanical unit. That is, a vertical and horizontal movement speed, a movable range, and the like of the bed limit a range where the bed can be moved in the vertical and horizontal directions within a time to move a bed from a point to the next point in the Z direction at a predetermined speed. Therefore, the X-ray CT apparatus of the present embodiment calculates a limit value of a movement amount in a direction orthogonal to the body-axis direction of the bed to control movement in the direction orthogonal to the body-axis direction of the bed based on a limit value.

Specifically, in the process of specifying a plurality of the control points P, a range that can be set as the control points (settable range) is set next. The settable range can be calculated by the computing device 30 according to the movement speed in the Z direction based on the vertical and horizontal movement speed, the movable range, and the like of the bed. Also, a setting value of the settable range may be included as a default value.

In a case where a next control point for a control point is specified within a movable range, it is set as the next control point. In a case where a next control point is specified beyond this range, re-specifying is requested without setting a control point, or it may be set so as to move to the closest position within this range. In case of having a problem with a control point that cannot be set if the cause is a horizontal and vertical movement speed of a bed, the problem can be improved by reducing a movement speed (i.e., a beam pitch) in the body-axis direction of the bed or reducing a scanner rotation speed, and these settings can also be changed. When the conditions are changed, a control point can be moved within a movable range even after the control point is set once.

In order to facilitate specifying a control point described above, a settable range may be displayed on a display device.

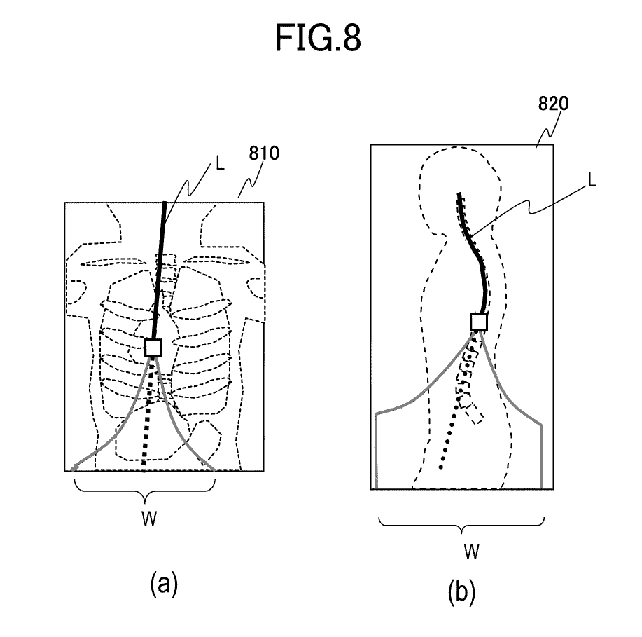

A display example is shown in FIG. 8. FIG. 8(a) shows a case where the coronal image 810 is displayed, and FIG. 8(b) shows a case where the sagittal image 820 is displayed. Although the settable range W is indicated by lines in FIG. 8, the indication method such as coloring the range is not limited to the example shown in FIG. 8. Although a case of displaying a two-dimensional image is shown in FIG. 8, the settable range is applied also to a case where a bed movement curve along a lumen is generated from a volume image. Thus, by displaying the settable range W, an operator can perform interactive bed movement curve setting smoothly.

After setting the bed movement curve thus, the bed 20 is moved in a direction orthogonal to the body-axis direction according to the set bed movement curve while moving the bed in the body-axis direction after scanning starts (Step S503). At this time, in a case where the bed movement measuring device 23 is provided, a bed movement trajectory on which the bed moved during scanning is measured as a bed movement curve. The measured bed movement trajectory is used for image reconstruction instead of the set bed movement curve in a case where there is a large error between a previously estimated bed movement curve and a bed movement curve on which the bed moved actually.

Lastly, an image is generated using projection data for each view detected by the X-ray detector 12 during scanning (S504). In the projection data for each view obtained in the above scanning (S503), an object position is shifted in the vertical and horizontal directions based on a bed movement curve. For example, when scanning is performed while moving a bed by 1 mm in the right direction for each view, an object in the center of the first view position will be shifted by 1 mm to the right side in the next second view. If such projection data is reconstructed as it is using a conventional image reconstruction method, artifacts due to an object motion and object shape distortion are caused. Therefore, in the present embodiment, reconstruction is performed while moving the reconstruction center position for each view according to the bed movement curve. That is, in the above example, the object position in the reconstruction image does not change as it is by shifting the reconstruction image by 1 mm to the right side in the second view. Hence, artifacts due to the vertical and horizontal movement of the bed and the object shape distortion can be improved.

Hereinafter, the specific image reconstruction computing method will be described in detail.

For comparison, an image reconstruction method of the conventional fan-beam mode supporting a helical scan will be first described. In the mage reconstruction of the conventional fan-beam mode, a tomographic image is generated by calculation using the following formula (2) for example.

$$\begin{matrix} [ Formula 2 ] & \\ I ( {x}_{I} , {y}_{I} , {z}_{I} ) = \frac{1}{2 π} {\int }_{- π}^{π} \frac{{R}^{2}}{{L ( β , {x}_{I} , {y}_{I} )}^{2}} {fP}_{fan} ( β , {α}_{I} , {v}_{I} ) d β & ( 2 ) \\ wherein , & \\ L ( β , {x}_{I} , {y}_{I} ) = \sqrt{{( R - {x}_{I} \cdot sin β + {y}_{I} \cdot cos β )}^{2} + {( {x}_{I} \cdot cos β + {y}_{I} \cdot sin β )}^{2}} & ( 2 \text{-} 1 ) \\ {α}_{I} = arc tan ( \frac{{x}_{I} \cdot cos β + {y}_{I} \cdot sin β}{R - {x}_{I} \cdot sin β + {y}_{I} \cdot cos β} ) & ( 2 \text{-} 2 ) \\ {v}_{I} = \frac{( {z}_{I} - {z}_{S} ) \cdot SID}{L ( β , {x}_{I} , {y}_{I} )} & ( 2 \text{-} 3 ) \\ {z}_{S} = \frac{T \cdot β}{2 π} + {z}_{S 0} & ( 2 \text{-} 4 )\end{matrix}$$

The definitions of the variables in the formula are as follows.

I: image data

x_{I}, y_{I}, and z_{I}: a target pixel position [mm]

L: a distance from the ray source to a target pixel [mm]

R: a distance between the ray source and the rotation center [mm]

β: projection angle of a fan beam [rad]

fP_{fan}: fan-beam projection data for filter correction

α_{I}: a channel angle (fan angle) [rad]

v_{I}: a detector row position [mm]

x_{S}, y_{S}, and z_{S}: a ray source position [mm]

SID: a distance from the ray source to the detector

T: a bed movement speed (Z direction) [mm/rotation]

On the contrary to the above, in the present embodiment, as values of x_{I} and y_{I} to be used in the above formulas (2-1) and (2-2), calculation of the formula (2) is performed using values to which movement amounts in the X and Y directions calculated from the bed movement curves κₓ and κ_{y} of the formula (1) were added. That is, the formulas (2-1) to (2-3) are changed as shown in the following formulas (3-1) to (3-3).

$$\begin{matrix} [ Formula 3 ] & \\ L ( β , {x}_{I} , {y}_{I} ) = \sqrt{\begin{matrix}{( R - ( {x}_{I} + {κ}_{x} ( β ) ) \cdot sin β + ( {y}_{I} + {κ}_{y} ( β ) ) \cdot cos β )}^{2} + \\ {( ( {x}_{I} + {κ}_{x} ( β ) ) \cdot cos β + ( {y}_{I} + {κ}_{y} ( β ) ) \cdot sin β )}^{2}\end{matrix}} & ( 3 \text{-} 1 ) \\ {α}_{I} = arc tan ( \frac{( {x}_{I} + {κ}_{x} ( β ) ) \cdot cos β + ( {y}_{I} + {κ}_{y} ( β ) ) \cdot sin β}{R - ( {x}_{I} + {κ}_{x} ( β ) ) \cdot sin β + ( {y}_{I} + {κ}_{y} ( β ) ) \cdot cos β} ) & ( 3 \text{-} 2 ) \\ {v}_{I} = \frac{( {z}_{I} - {z}_{S} ) \cdot SID}{L ( β , {x}_{I} , {y}_{I} )} & ( 3 \text{-} 3 )\end{matrix}$$

In the reconstruction of the present embodiment, it is found that the pixel positions x and y has been corrected by the vertical and horizontal position data κₓ and κ_{y} of a bed comparing to the conventional reconstruction. The vertical and horizontal position data κₓ and κ_{y} of the bed are view functions, and that is, it is found that this is equal to that the reconstruction center is moved for each view.

Although the formula (2) is the image reconstruction method of the fan-beam mode, a method using the fan-parallel conversion that converts from fan-beam projection to parallel-beam projection is used in order to increase a computation speed and improve image quality homogeneity. Image reconstruction of the conventional parallel-beam mode can be expressed in the following formula (4) for example.

$$\begin{matrix} [ Formula 4 ] & \\ I ( {x}_{I} , {y}_{I} , {z}_{I} ) = \frac{1}{π} {\int }_{{B}_{s} ( {x}_{I} , {y}_{I} , {z}_{I} )}^{{B}_{e} ( {x}_{I} , {y}_{I} , {z}_{I} )} {fP}_{para} ( φ , {t}_{I} , {v}_{I} ) \cdot W ( φ - {B}_{s} ( {x}_{I} , {y}_{I} , {z}_{I} ) - F π ) \cdot d φ & ( 4 ) \\ wherein , & \\ {v}_{I} ( {x}_{I} , {y}_{I} , {z}_{I} , φ ) = \frac{( {z}_{I} - {z}_{S} ( {x}_{I} , {y}_{I} , φ ) ) \cdot SID}{L ( {x}_{I} , {y}_{I} , φ )} & ( 4 \text{-} 1 ) \\ {z}_{S} ( {x}_{I} , {y}_{I} , φ ) = \frac{T \cdot ( φ + arc sin ( \frac{{t}_{I} ( {x}_{I} , {y}_{I} , φ )}{R} ) )}{2 π} + {z}_{S 0} & ( 4 \text{-} 2 ) \\ L ( {x}_{I} , {y}_{I} , φ ) = D ( {x}_{I} , {y}_{I} , φ ) + w ( {x}_{I} , {y}_{I} , φ ) & ( 4 \text{-} 3 ) \\ D ( {x}_{I} , {y}_{I} , φ ) = \sqrt{{R}^{2} - {t}_{I}^{2} ( {x}_{I} , {y}_{I} , φ )} & ( 4 \text{-} 4 ) \\ w ( {x}_{I} , {y}_{I} , φ ) = - {x}_{I} \cdot sin ( φ ) + {y}_{I} \cdot cos ( φ ) & ( 4 \text{-} 5 ) \\ {t}_{I} ( {x}_{I} , {y}_{I} , φ ) = {x}_{I} \cdot cos ( φ ) + {y}_{I} \cdot sin ( φ ) & ( 4 \text{-} 6 )\end{matrix}$$

The definitions of the variables in the formula are as follows (the symbols same as those used in the above formula have the same definitions, and the explanations are omitted).

fPₚₐᵣₐ: parallel-beam projection data for filter correction

φ: a projection angle of the parallel beam [rad]

Wₚ(φ): a view weight for the parallel beam

F: a phase width for which back projection is performed (an angle width of a view in which back projection is performed for a pixel)

Also, the rearrangement process from fan-beam projection to parallel-beam projection (fan-parallel conversion) is expressed as the following formula (5) for example.

[Formula 5]

Pₚₐᵣₐ(φ,t)=P_{fan}(β−α,R sin α) (5)

φ=β−α (5-1)

t=R·sin α (5-2)

The reconstruction filtering process is expressed as the following formulas (6-1) and (6-2) for example.

[Formula 6]

fP_{fan}(β,α,ν)=∫_{-α}_{ₘ}^{α}^{ₘ}P_{fan}(β,α′,ν)·g(α−α′,ν)·da′ (6-1)

fPₚₐᵣₐ(φ,t,ν)=∫_{-α}_{ₘ}^{α}^{ₘ}Pₚₐᵣₐ(φ,t′,ν)·g(t−t′,ν)·dt′ (6-2)

For the present embodiment, in case of applying the parallel-beam reconstruction using the above formula (4), calculation of the formula (4) is performed by converting the values x_{I} and y_{I} included in the two members of the formula (4-3) to calculate the distance L from the ray source to a target pixel into values to which movement amounts in the X and Y directions to be calculated from the bed movement curves κₓ and κ_{y} of the formula (1) were added. That is, the parallel-beam reconstruction is performed using the following formulas (7) and (7-1) to (7-8).

$$\begin{matrix} [ Formula 7 ] & \\ I ( {x}_{I} , {y}_{I} , {z}_{I} ) = \frac{1}{π} {\int }_{{B}_{s} ( {x}_{I} , {y}_{I} , {z}_{I} )}^{{B}_{e} ( {x}_{I} , {y}_{I} , {z}_{I} )} {fP}_{para} ( φ , {t}_{I} , {v}_{I} ) \cdot W ( φ - {B}_{s} ( {x}_{I} , {y}_{I} , {z}_{I} ) - F π ) \cdot d φ & ( 7 ) \\ wherein , & \\ {v}_{I} ( {x}_{I} , {y}_{I} , {z}_{I} , φ ) = \frac{( {z}_{I} - {z}_{S} ( {x}_{I} , {y}_{I} , φ ) ) \cdot SID}{L ( {x}_{I} , {y}_{I} , φ )} & ( 7 \text{-} 1 ) \\ {z}_{S} ( {x}_{I} , {y}_{I} , φ ) = \frac{T \cdot ( φ + arc sin ( \frac{{t}_{I} ( {x}_{I} , {y}_{I} , φ )}{R} ) )}{2 π} + {z}_{S 0} & ( 7 \text{-} 2 ) \\ L ( {x}_{I} , {y}_{I} , φ ) = D ( {x}_{I} , {y}_{I} , φ ) + w ( {x}_{I} , {y}_{I} , φ ) & ( 7 \text{-} 3 ) \\ D ( {x}_{I} , {y}_{I} , φ ) = \sqrt{{R}^{2} - {t}_{I}^{2} ( {x}_{I} , {y}_{I} , φ )} & ( 7 \text{-} 4 ) \\ w ( {x}_{I} , {y}_{I} , φ ) = - ( {x}_{I} + {κ}_{x} ( {β}^{( n )} ) ) \cdot sin ( φ ) + ( {y}_{I} + {κ}_{y} ( {β}^{( n )} ) ) \cdot cos ( φ ) & ( 7 \text{-} 5 ) \\ {t}_{I}^{( n )} ( {x}_{I} , {y}_{I} , φ ) = ( {x}_{I} + {κ}_{x} ( {β}^{( n )} ) ) \cdot cos ( φ ) + ( {y}_{I} + {κ}_{y} ( {β}^{( n )} ) ) \cdot sin ( φ ) & ( 7 \text{-} 6 ) \\ {β}^{( n )} = φ + α = φ + arc sin \frac{{t}^{( n - 1 )}}{R} & ( 7 \text{-} 7 ) \\ {t}_{I}^{( 0 )} = {x}_{I} \cdot cos φ + {y}_{I} \cdot sin φ & ( 7 \text{-} 8 ) \\ n = 1 , 2 , 3 … & \end{matrix}$$

Additionally, although the above formulas (7-3) and (7-4) are apparently the same as the conventional parallel-beam reconstruction formulas (4-3) and (4-4), the pixel positions x and y used in the formulas (7-3) and (7-4) are corrected by κₓ and κ_{y} of the vertical and horizontal bed position information respectively. That is, κₓ and κ_{y} of the vertical and horizontal bed position information are a view function, and it is found that this is equivalent to that the reconstruction center is moved for each view. Also, the above formulas are a circulation function in which a parallel-beam projection angle φ and the pixel positions x and y are parameters, and the sufficient number of circulations of this circulation function (the number of loops) is only a few times.

Although the image reconstruction method in a case of a helical scan was described above using the formulas, the similar image reconstruction can be performed by changing a coordinate of the reconstruction center position used for reconstruction for each view according to the bed movement curve also in a case of a different scan mode. Hence, an image without artifacts caused by moving the bed vertically and horizontally can be acquired.

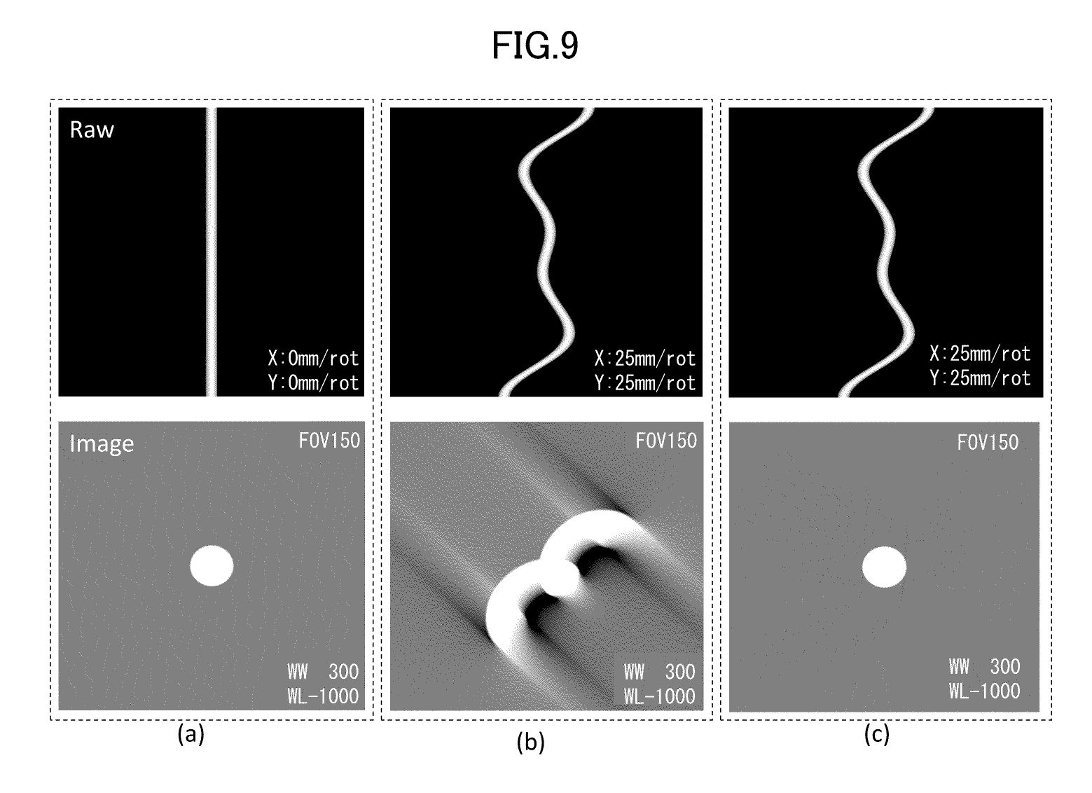

FIG. 9 shows the results where the above image reconstruction is performed for the narrow cylindrical phantoms. The three diagrams on the upper side in FIG. 9 are projection data, and those on the lower side are tomographic images after the image reconstruction. (a) shows a case of scanning without moving a bed, (b) shows a case of performing conventional reconstruction by scanning while moving the bed in the X and Y directions, and (c) shows a case of performing the image reconstruction of the present embodiment by scanning while moving the bed in the X and Y directions. In a case of scanning while moving the bed, the projection data is that in which a motion in the scanning center is reflected as shown on the upper side of (b) and (c).

If the projection data is reconstructed as it is, artifacts appear as shown in (b), which causes image distortion. On the contrary to this, in a case of performing reconstruction of the present embodiment, a quality image can be acquired without artifacts and distortion similarly to the scanning (a) without moving a bed.

According to the present embodiment, provided is an X-ray CT apparatus that acquires an image which does not have artifacts caused by moving a bed and which has high spatial resolution of a site of interest of an object by scanning it while moving the bed so that the site is located approximately in the scanning center.

### Second Embodiment

Although changing the scanning center position according to the site of interest in the present embodiment is similar to the first embodiment, the present embodiment is characterized by that a bed position is not moved; but a tilt angle of the scanner is changed according to the tilt variation of the site of interest. Hereinafter, focusing on the differences from the first embodiment, the present embodiment will be described.

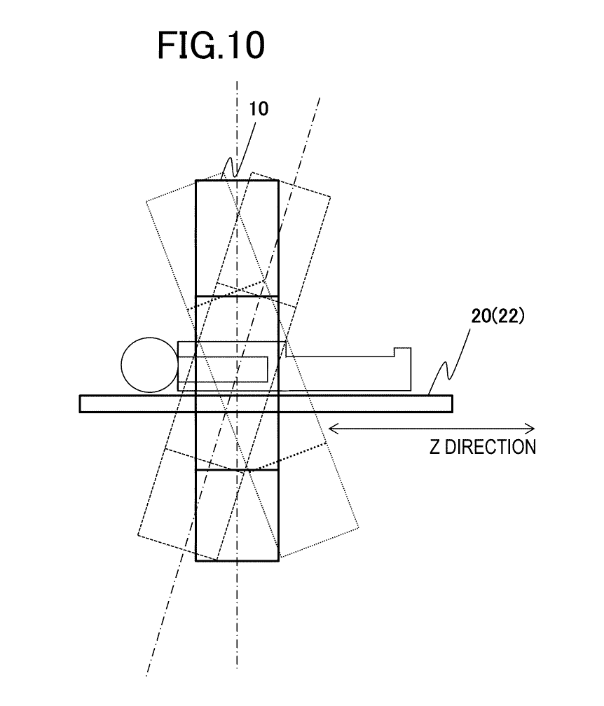

As shown in FIG. 10, the basic posture of the scanner 10 is perpendicular so that a straight line connecting the X-ray source of the X-ray generation device 11 to the center of the X-ray detector is orthogonal to the bed movement direction (Z direction). The scanner 10 can take a tilted posture from the perpendicular posture in a range where the top plate 22 of the bed 20 moving in the scanner opening does not interfere with the opening. For example, in a case of scanning in the helical scan mode in the tilted posture, spatial resolution in the Z direction can be improved more than a perpendicular posture, and the helical scan mode in a tilted posture has been well known. However, if a tilt angle is changed during the scan, this generates an error during image reconstruction, and great artifacts are generated. Therefore, the tilt angle has been fixed. In the present embodiment, a tilt angle is changed according to the shape of a site of interest during scanning in order to continue scanning with high spatial resolution for the site of interest.

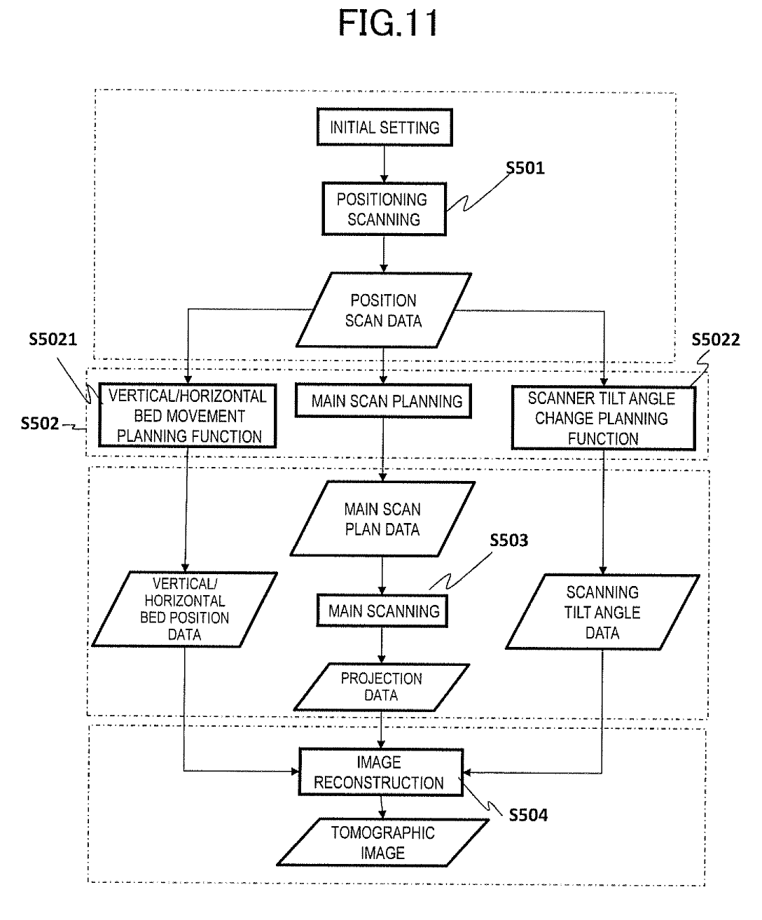

Hereinafter, referring to the scanning procedure shown in FIG. 11, operations of the X-ray CT apparatus of the present embodiment will be described. Also in FIG. 11, the same symbols are shown in the steps corresponding to the scanning procedure shown in FIG. 5.

Also in the present embodiment, performing pre-scanning (S501) and setting a bed movement curve based on a pre-scan image (S5021) are the same as the first embodiment, and a positioning image is used for the pre-scan image. The present embodiment further calculates an angle change amount ΔΓ for the Z direction of the set bed movement curve in order to determine a tilt angle change amount. The angle change amount ΔΓ can be calculated using the formula 8 for example.

[Formula 8]

ΔΓ=arctan((κ_{y}(β)−κ_{y}(β−Δβ))/Δβ) (8)

Δβ indicates a range of a predetermined view angle.

A scanner tilt angle γ is expressed as “γ=−Γ” when setting a rotation in the positive direction of the Z axis from that of the Y axis as “+” and the reverse rotation as “−”. Hence, a tilt angle γ is calculated as a function of the view angle β (S5022).

At this time, a tiltable range of the scanner can be set just as a movable range is set in the first embodiment. As described above, the tilt angle is limited to a range where the top plate of the bed does not interfere with the opening. Also, when the top plate is moved vertically and horizontally, the tiltable range changes. Because a tiltable range (the maximum angle in both the directions of ±) is determined according to the vertical and horizontal position of the bed, the computing device determines whether or not an angle calculated by the above formula (8) is an allowable angle in a bed position (a position in a case of moving in a set movement amount) in the view. If the angle exceeds the allowable angle, it is set to an allowable maximum angle.

The set tilt angle will be used for image reconstruction later. Also, in a case where a device that measures and records a scanner tilt angle is provided, the angle information from the recording device is used for image reconstruction. Hence, even if there is an error between the set tilt angle and an actual tilt angle, image reconstruction without an error can be performed.

Thus, after setting a tilt angle change amount, main scanning starts, and then the bed 20 moves in the Z direction to perform scanning while changing a tilt angle of the scanner 10 according to the view angle (S503). Additionally, in a case where movement amounts of the bed 20 in the X and Y directions are set by a bed movement curve, the bed may be moved in the vertical and horizontal directions as a scanner tilt angle changes. For example, various types of scan modes shown in FIG. 3 can be adopted also here.

After scanning, reconstruction is performed using projection data collected by the X-ray detector 12 (S504). For example, the reconstruction can be performed using the following formula (9) in the fan-beam mode.

$$\begin{matrix}[ Formula 9 ] & \\ I ( {x}_{I} , {y}_{I} , {z}_{I} ) = \frac{1}{2 π} {\int }_{- π}^{π} \frac{{R}^{2}}{{L ( β , {x}_{I} , {y}_{I}^{′} )}^{2}} {fP}_{fan} ( β , {α}_{I} , {v}_{I} ) d β & ( 9 ) \\ wherein , & \\ L ( β , {x}_{I} , {y}_{I}^{′} ) = \sqrt{\begin{matrix}{( R - ( {x}_{I} + {κ}_{x} ( β ) ) \cdot sin β + {y}_{I}^{′} \cdot cos β )}^{2} + \\ {( ( {x}_{I} + {κ}_{x} ( β ) ) \cdot cos β + {y}_{I}^{′} \cdot sin β )}^{2}\end{matrix}} & ( 9 \text{-} 1 ) \\ {α}_{I} = arc tan ( \frac{( {x}_{I} + {κ}_{x} ( β ) ) {x}_{I} \cdot cos β + {y}_{I}^{′} \cdot sin β}{R - ( {x}_{I} + {κ}_{x} ( β ) ) \cdot sin β + {y}_{I}^{′} \cdot cos β} ) & ( 9 \text{-} 2 ) \\ {v}_{I} = \frac{( {z}_{I} - {z}_{S}^{′} ) \cdot SID}{L ( β , {x}_{I} , {y}_{I}^{′} )} & ( 9 \text{-} 3 ) \\ {z}_{S}^{′} = \frac{cos γ ( β ) \cdot T ( β ) \cdot β}{2 π} + {z}_{S 0} & ( 9 \text{-} 4 ) \\ {y}_{I}^{′} = {y}_{I} + {κ}_{y} ( β ) + γ {y}_{I} = {y}_{I} + {κ}_{y} ( β ) + \frac{sin γ ( β ) \cdot T ( β ) \cdot β}{2 π} & ( 9 \text{-} 5 )\end{matrix}$$

Although the above formulas (9) and (9-1) to (9-3) correspond to (2) and (2-1) to (2-3) of the first embodiment, a target pixel position in the Y direction “y_{I}” used in these formulas is changed to “y′_{I}” shown in the formula (9-5) because the scanner is tilted. Also, the ray source position in the Z direction “Z_{S}” used in the formula (2-3) is changed to “Z′_{S}” shown in the formula (9-4).

For image reconstruction in the parallel-beam mode, a scanner tilt angle change is reflected, and then the formulas (7) and (7-1) to (7-8) are changed as follows. Hence, even when a scanner tilt angle is changed during scanning, no error is generated, and an image without artifacts can be acquired.

$$\begin{matrix} [ Formula 10 ] & \\ I ( {x}_{I} , {y}_{I} , {z}_{I} ) = \frac{1}{π} {\int }_{{B}_{s} ( {x}_{I} , {y}_{I} , {z}_{I} )}^{{B}_{e} ( {x}_{I} , {y}_{I} , {z}_{I} )} {fP}_{para} ( φ , {t}_{I}^{( n )} , {v}_{I} ) \cdot W ( φ - {B}_{s} ( {x}_{I} , {y}_{I} , {z}_{I} ) - F π ) \cdot d φ & ( 10 ) \\ wherein , & \\ {v}_{I} ( {x}_{I} , {y}_{I} , {z}_{I} , φ ) = \frac{( {z}_{I} - {z}_{S} ( {x}_{I} , {y}_{I} , φ ) ) \cdot SID}{L ( {x}_{I} , {y}_{I} , φ )} & ( 10 \text{-} 1 ) \\ {z}_{S} ( {x}_{I} , {y}_{I} , φ ) = \frac{cos γ ( {β}^{( n )} ) \cdot T ( {β}^{( n )} ) \cdot ( φ + arc sin ( \frac{{t}_{I}^{( n )} ( {x}_{I} , {y}_{I} , φ )}{R} ) )}{2 π} + {z}_{S 0} & ( 10 \text{-} 2 ) \\ L ( {x}_{I} , {y}_{I} , φ ) = D ( {x}_{I} , {y}_{I} , φ ) + {w}_{I} ( {x}_{I} , {y}_{I} , φ ) & ( 10 \text{-} 3 ) \\ D ( {x}_{I} , {y}_{I} , φ ) = \sqrt{{R}^{2} - {( {t}_{I}^{( n )} ( {x}_{I} , {y}_{I} , φ ) )}^{2}} & ( 10 \text{-} 4 ) \\ {w}_{I} ( {x}_{I} , {y}_{I} , φ ) = - ( {x}_{I} + {κ}_{x} ( {β}^{( n )} ) ) \cdot sin φ + ( ( {y}_{I} + {κ}_{y} ( {β}^{( n )} ) ) + Δ {y}_{I}^{( n - 1 )} ) \cdot cos ( φ ) & ( 10 \text{-} 5 ) \\ {t}_{I}^{( n )} ( {x}_{I} , {y}_{I} , φ ) = ( {x}_{I} + {κ}_{x} ( {β}^{( n )} ) ) \cdot cos φ + ( ( {y}_{I} + {κ}_{y} ( {β}^{( n )} ) ) + Δ {y}_{I}^{( n - 1 )} ) \cdot sin φ & ( 10 \text{-} 6 ) \\ Δ {y}_{I}^{( n )} ( {x}_{I} , {y}_{I} , φ ) = \frac{sin γ ( {β}^{( n )} ) \cdot T ( {β}^{( n )} ) \cdot ( φ + arcsin ( \frac{{t}_{I}^{( n - 1 )} ( {x}_{I} , {y}_{I} , φ )}{R} ) )}{2 π} & ( 10 \text{-} 7 ) \\ {β}^{( n )} = φ + α = φ + arctan \frac{{t}_{I}^{( n - 1 )}}{R} & ( 10 \text{-} 8 ) \\ {t}_{I}^{( 0 )} = {x}_{I} \cdot cos φ + {y}_{I} \cdot sin φ & ( 10 \text{-} 9 ) \\ n = 1 , 2 , 3 … & \end{matrix}$$

According to the present embodiment, even if a site of interest of an object is bent (this is found in many cases) or even if there is some angle to the Z direction although the site of interest is along the body-axis direction (Z direction), a satisfactory image can be acquired because the orthogonal cross section can be always scanned according to the bending degree or the angle. Also, although a tilt angle is changed, an image without artifacts can be acquired.

Additionally, although the above formulas (9) and (10) show general the formulas including vertical and horizontal movements of a bed in addition to a tilt angle change, the present embodiment includes a case of changing a tilt angle only. That is, the operation procedure in FIG. 11 comprehensively shows the operation procedures of the first and second embodiments, the present embodiment is characterized by performing Step S5022 on the right side of Step S502 in the diagram, and cases of performing and not performing Step S5021 on the left side are included in the present embodiment. Incidentally, the first embodiment is the case of performing Step S5021 on the left side.

### Third Embodiment

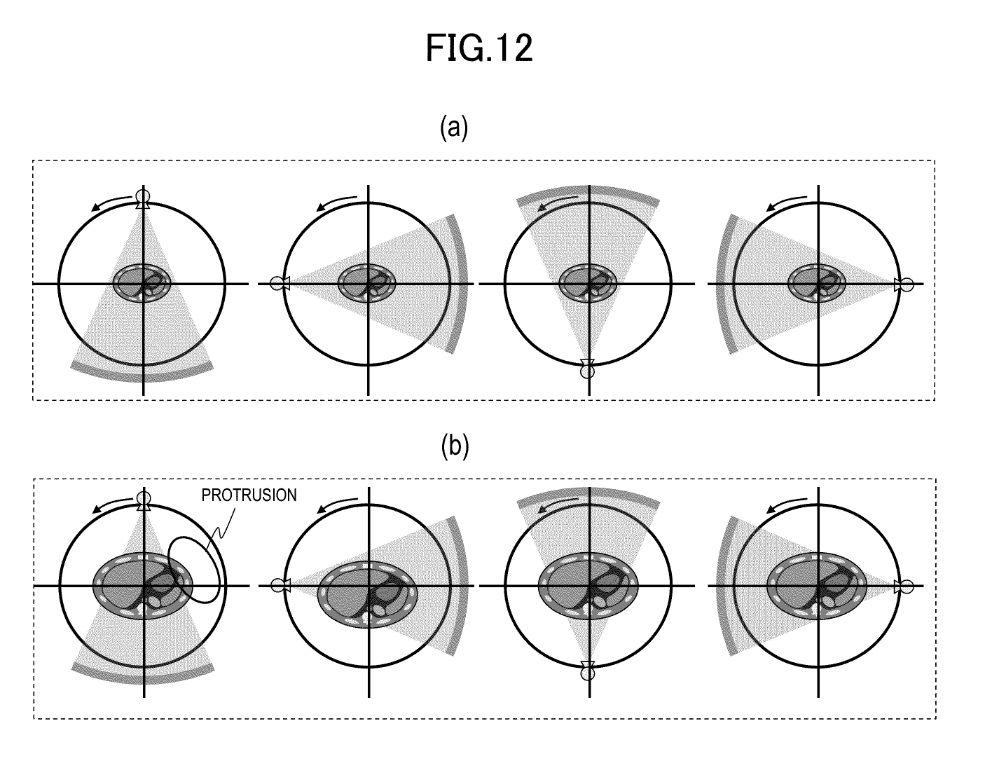

The present embodiment is characterized by setting a bed movement range so as to cover an object by enlarging an FOV when the object is large comparing to the size of the X-ray detector 12. First, the relationship between the sizes of the object and the X-ray detector will be described referring to FIG. 12. FIG. 12(a) shows a case where the detector size is large enough to cover the object entirely, and FIG. 12(b) shows a case where the object cannot be entirely covered because the detector size is small.

As shown in FIG. 12(a), in a case where a detector size is relatively large, an object enters within an angle range of the fan beam generated from the X-ray generation device 11, and projection data required for tomographic image generation can be collected by a rotation of 360 (180 at minimum) degrees. On the other hand, as shown in FIG. 12(b), in a case where a detector size is relatively small, an object partially protrudes out of the fan beam, and the 360-degree rotation does not provide projection data required for a tomographic image, which cannot reconstructs an image properly.

On the contrary to this, there is a method to acquire an image by scanning twice in different positions in the horizontal direction of a bed and synthesizing image data acquired by the two times of scanning, but in this case, there is a possibility that an image is blurred or that artifacts appear if an object posture changes during the two times of scanning. Also, there is a case where simply scanning in different positions does not work properly. The X-ray CT apparatus of the present embodiment can enlarge a substantial FOV even in a case where a detector size is relatively small by scanning sequentially while changing a bed (object) position and performing image reconstruction using bed position information, which can scan a large-size object.

Hereinafter, operations of the X-ray CT apparatus of the present embodiment will be described again referring to the scanning procedure shown in FIG. 5.

First, a pre-scan is performed (S501), the pre-scanned image is displayed on the display device, and then a range for performing a bed movement scan by the present embodiment is determined. The bed movement scan is to perform scanning while moving a bed in the vertical and/or horizontal directions. For example, in a case of scanning an object entirely from the head to the foot, there is a possibility that the chest and the abdomen cannot be covered entirely, though the detector can entirely cover the head, the foot, and the like as shown in FIG. 12(a). In such a case, slices to start and end the bed movement scan are specified in order to determine a range for performing the bed movement scan (a range in the Z direction). The bed movement scan may be performed for the entire scanning range without specifying a range for the bed movement scan, and the range determination process is omitted in this case.

Next, a movement range in the vertical and horizontal directions of a bed for bed movement scanning is determined to set a bed movement curve (S502). The movement range in the vertical and horizontal directions of a bed may be set to a predetermined movement range based on a previously-measured abdominal circumference of an object, a height from the upper surface of the top plate to that of the object, and the like or may be specified on a tomographic image by displaying a previously-scanned tomographic image of a slice within a range for the bed movement scanning on the display device. Also, because there are mechanical limitations for the bed movement range, the movement range is set within the limitation range.

After the movement range is specified, the number of views in the movement range is set to determine a bed movement curve. It is desirable that the number of views is equal to or more than M×1.5 when the number of views for 360 degrees is M. Hence, an image in which no data is missing can be acquired. The bed movement curve is expressed by a straight line (the formulas (11-1) and (11-2)) that linearly changes a bed position from one point (one end of the movement range) to the other point (the other end of the movement range) in the vertical and horizontal directions for a view.

[Formula 11]

κₓ(β)=aβ+x0 (11-1)

κ_{y}(β)=bβ+y0 (11-2)

In the formulas, a and b represent constants, and x0 as well as y0 represent coordinates of the scanning center at the time of starting bed movement scanning.

Next, scanning is performed while moving a bed based on the set bed movement curve (S503). Any of the scan modes shown in FIG. 3 may be used, and in case of the normal scan, the bed movement scanning is performed by the specified number of views in each slice position where the movement in the Z direction stops while intermittently moving a bed in the Z direction. In case of the helical scan, the bed movement (movement in the X and Y directions) scanning is performed while continuously moving the bed in the Z direction. This is similar also in case of the shuttle scan. When a range for the bed movement scanning is set, the bed movement scanning is performed only for the set range, and the scanning is performed in the XY direction in a state where the bed is fixed outside the range.

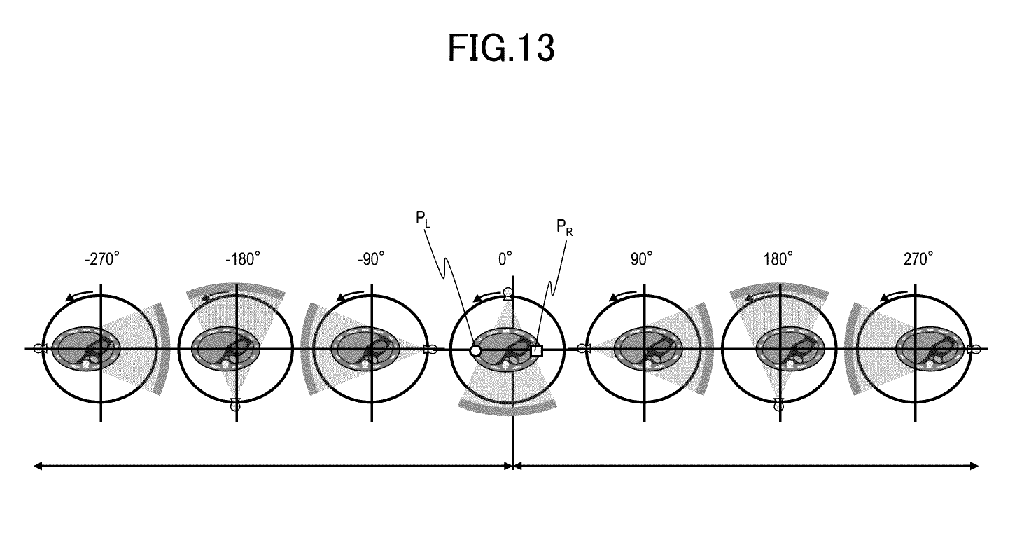

An example of a case where bed movement scanning is performed for a cross section is shown in FIG. 13. Although FIG. 13 shows an example of moving a bed only in the horizontal direction (X direction), the direction for moving the bed may be the vertical direction or a direction where the vertical and horizontal directions were synthesized. As shown in the diagram, the scanning center moves to the right for each view from a position more left than the object center as the bed moves.

Although there is a part where an object protrudes out of the expansion of a fan beam that the X-ray detector 12 receives in an individual view, the required data for CT image reconstruction is acquired from all the regions of the object by rotating the scanner (rotary disk) by one and a half rounds. Specifically, when the scanner position in the central portion of FIG. 13 is set as 0 degree, projection data to be used for reconstruction of an object part including the point P_{R} on the right end of the object is acquired in a view from −270 degrees (the left end) to 0 degree, and projection data to be used for reconstruction of an object part including the point P_{L} on the left end of the object is acquired in a view from 0 degree to +270 degree (the right end).

Additionally, although FIG. 13 shows a case of rotating the scanner by one and a half rounds, the double amount of the above projection data may be acquired by rotating the scanner by two rounds. That is, scanning starts in a bed position where the object center is approximately the scanning center, the bed starts movement to the left at the scanner angle of −360 degrees (0 degree), and the bed position returns after reaching the left end at the scanner angle of −270 degrees (the left end of FIG. 13). Similarly, the bed position returns at the right end at the scanner angle of +270 degrees and gets back to the same bed position (where the object center is approximately the scanning center) as 0 degree at 360 degrees.

After scanning, the former half and the latter half of these projection data of one and a half or two rounds are used for reconstruction to synthesize the two images and generate a tomographic image (S504). Similarly to the first embodiment, the fan-beam reconstruction is perform using the formula (2) corrected by the formulas (3-1) and (3-2), and the parallel-beam reconstruction is performed using the formula (7). However, as the bed movement curve κ, the bed movement curve shown in the formula (11) is used. Alternatively, in a case where the bed movement measuring device 23 is provided, reconstruction is performed using a bed movement trajectory (position information) of actual movement during scanning.

Thus, because correction is made according to the bed movement amount for a pixel position in image reconstruction, an image without artifacts can be acquired.

According to the present embodiment, a satisfactory image can be acquired for the entire object without missing data by moving a bed position (object position) so as to acquire projection data covering the entire object during scanning even when a part of the object protrudes out of a fan beam because the X-ray detection device size is small comparing to the object size. Also, in comparison to a case of scanning multiple times in different bed positions, the present embodiment performs scanning sequentially, is not affected by a posture change of the object between scanning steps, and does not have an image quality deterioration problem caused by the posture change.

### Fourth Embodiment

The present embodiment is characterized by that a sampling density to be determined according to the detector size is increased to improve spatial resolution by adopting bed movement scanning.

Generally, a sampling pitch of a fan beam is determined according to the detector size (element size). Although a sampling density can be increased because a sampling pitch becomes small when a size of elements comprising the detector is small, an occupation rate of a separator preventing crosstalk between elements is increased when the element size is small, which lowers a dose efficiency. Therefore, the element size of a general CT apparatus is approximately 1 mm in the channel and detector row directions respectively, and the sampling pitch is approximately 0.6 mm near the scanning center. Also, in case of using a detector that offsets a detector channel referred to as “quarter offset” by a one-fourth channel, spatial resolution of approximately 0.35 mm is achieved near the rotation center by setting so as to shift a sampling position for each counter data.

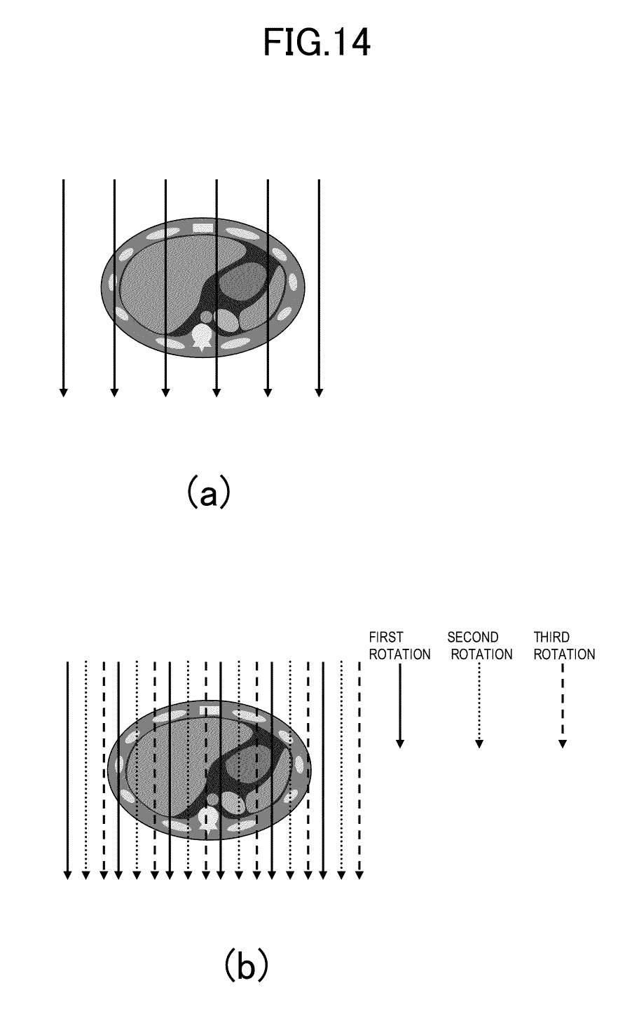

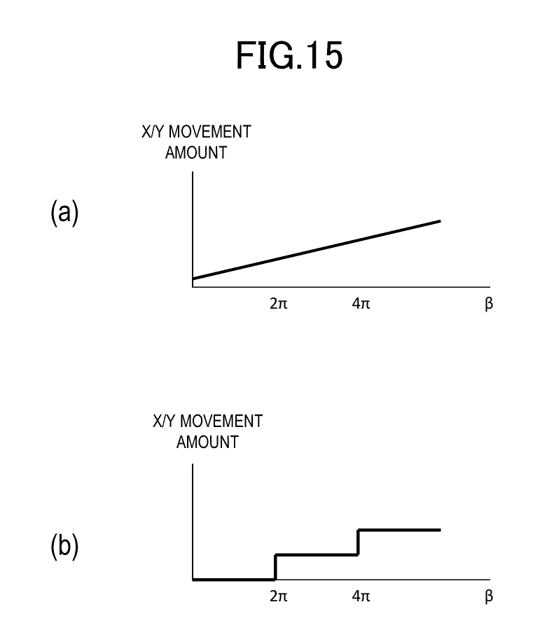

The present embodiment achieves spatial resolution equal to or greater than that of a quarter offset detector by adopting bed movement scanning. Hereinafter, the details of the present embodiment will be described referring to FIGS. 14 and 15. Also, the processing procedure refers to the scanning procedure shown in FIG. 5 as needed.

Although a bed moves in the vertical and/or horizontal direction during scanning also in the present embodiment similarly to the above embodiments, setting a bed movement curve using a pre-scanned image can be omitted, and the bed movement curve is determined by considering a sampling pitch in the scanning center in the present embodiment (S501). As an example, the description will be made taking a case of reducing the sampling pitch to one third.

Although a sampling pitch is a predetermined pitch (referred to as “basic pitch”) to be determined according to the detector size as shown in FIG. 14(a) in a case where there is no bed movement, it is configured so that the same slice is scanned three times at the same projection angle by scanning the same slice during three rounds in the present embodiment.

At this moment, a bed position is shifted in the X direction for example so that the scanning center is shifted one third from the basic pitch for each rotation. Hence, the sampling pitch becomes one third of the basic pitch case as shown in FIG. 14(b) at the end time of three-time scanning, that is, the sampling density becomes triple, which can improve spatial resolution.

A bed movement curve may be set so as to be moved continuously for one rotation of the scanner as shown in FIG. 15(a) or so as to move a bed position for each rotation as shown in FIG. 15(b). In this case, a change amount of the bed movement for each rotation is not one third of the basic pitch but may be set to “an integer multiple of the basic pitch+one-third pitch”. Hence, in case of the bed movement curve of FIG. 15(a) for example, a relatively large change amount can be set as a change amount from the first view of the first rotation to the last view of the third rotation, which can easily control the mechanical unit of the bed.

Also, although only the movement amount in the X direction is shown in FIG. 14, the bed movement direction may be the Y direction or both the X and Y directions.

Next, based on the bed movement curve, scanning is executed while moving a bed (S503). Scanning the same slice is performed during three rounds as described above. The scan mode may be any of the normal scan, accused helical scan, and shuttle scan. Three projection data whose scanning center positions are different can be acquired by scanning. These three projection data are, for example, a function comprised of a view β, a channel angle (fan angle) αI, and a detector row position v_{I} as shown in the following formula (12).

[Formula 12]

P_{fan}(β,α_{I},ν_{I}) (12)

For each projection data, the values calculated using the above formulas (3-1) to (3-3) is substituted for αI and vI of the formula (12), and projection data sampled at a high density can be acquired by synthesizing these three projection data.

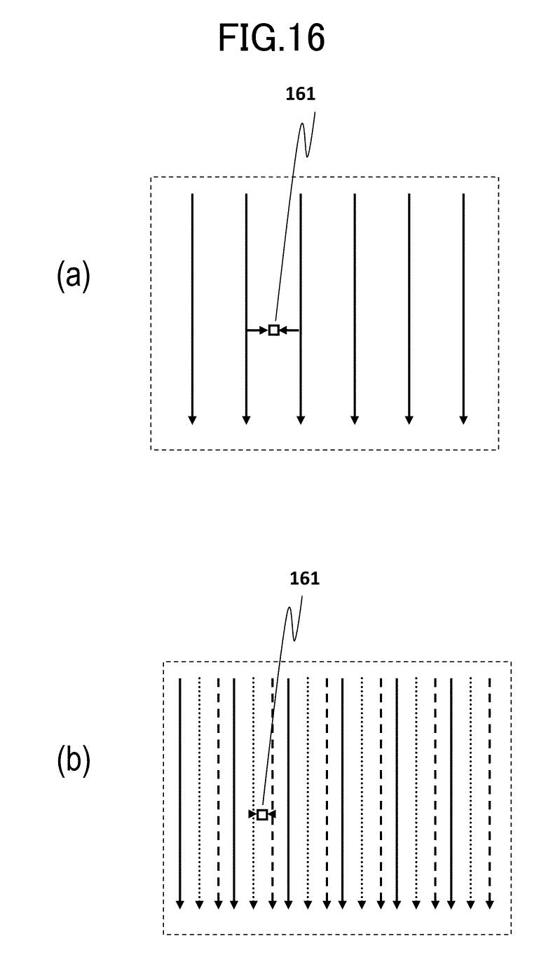

Image reconstruction after scanning is performed using data closest to a predetermined pixel as the data of the said pixel from among the data sampled at a high density by the multiple scans (S504). FIG. 16 shows the above image reconstruction. FIG. 16(a) is a diagram explaining a conventional reconstruction, and FIG. 16(b) is a diagram explaining the reconstruction of the present embodiment. Additionally, although raw projection data is fan-beam-shaped projection data when seen from the body-axis direction, a parallel-beam case is shown here to simplify the description because a fan beam can be converted into a parallel beam by fan-parallel conversion.

As shown in the diagram, data of a predetermined pixel (in the position shown using a square in the diagram) 161 is interpolated using projection data close to the data in reconstruction. In the present embodiment, it is understood that the data of the pixel 161 interpolated using the closest data is highly accurate compared to data interpolated using projection data of a conventional method. Then, in case of parallel-beam reconstruction, the formula (7) or (4) is used for the reconstruction. In this case, it is desirable to use a reconstruction filter with a wider band than that used for conventional reconstruction whose spatial resolution is low. Hence, an image of high spatial resolution can be reconstructed without hindering the effect of high-density sampling.

According to the present embodiment, data sampled at a high density can be acquired while scanning sequentially without changing a detector or the like of a conventional apparatus, which can improve spatial resolution of an image. Additionally, although a sampling density can be increased theoretically by performing multiple scans with the scanning center position shifted by one half, one third, . . . etc. of an element size (an array pitch), it is difficult to accurately move a bed in a movement amount smaller than the element size. On the contrary to this, in the present embodiment, a movement amount equal to or more than the element size is set as the maximum movement amount of the bed, and the bed is only moved continuously to the maximum movement amount, which can simplify bed control.

### Fifth Embodiment

The present embodiment also adopts bed movement scanning in order to increase a sampling density to be determined according to the detector size and improve spatial resolution similarly to the fourth embodiment. However, while the X-ray CT apparatus of the fourth embodiment has a function to highly densify and reconstruct projection data, the X-ray CT apparatus of the present embodiment is different in reconstructing and synthesizing a plurality of images whose sampling positions were shifted. Hereinafter, the different points will be mainly described.

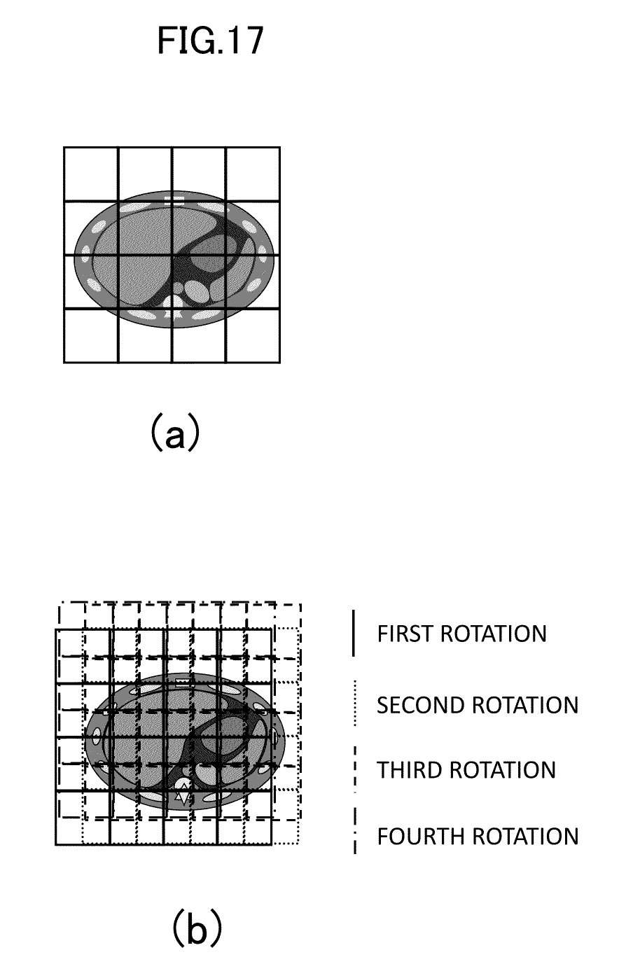

In the present embodiment, as shown in FIG. 17(b) for example, scanning is performed for the same slice during four rounds in order to acquire four images whose scanning center positions are different. A bed movement amount is determined so that the movement amount in the vertical and horizontal directions per rotation corresponds to a shift amount of the image (S502). The shift amount of the image is, for example, a shift amount at equal intervals so that a sampling position of an image is halfway to a sampling position of the other image. In the example shown in FIG. 17, a bed movement amount is determined so that the second round position is shifted by one half pitch in the right direction from the first pitch position; the third round position is shifted by one half pitch in the upper direction from the second pitch position; and the fourth round position is shifted by one half pitch from the third pitch position (i.e., a position shifted in the upper round from the first round position). The bed movement amount may be set so as to be changed in each view of each round or so as to be fixed in each round (refer to FIG. 15 of the fourth embodiment).

Next, scanning is performed based on a determined bed movement amount (S503), and projection data for each round is acquired. These projection data is reconstructed to acquire an image (S504). The formula (2) or the like can be used for fan-beam reconstruction, and the formula (4) or the like can be used for parallel-beam reconstruction.

In case of continuously moving a bed during one round at this time, the formula (3), (7), or the like is used for correction using bed position information. The bed position information may be a movement amount set in S502, and a bed movement trajectory (position information) on which a bed moved actually during scanning is used in a case where the bed movement measuring device 23 is provided.

Next, the acquired four images are used for generating one image. When pixel sampling positions for an object are shifted at equal intervals at this time, one image can be generated without interpolation processing. Spatial resolution of each reconstruction image before synthesization is the same as that of a normally scanned image shown in FIG. 17(a). Therefore, although the number of pixels of the synthesized image is four times as great as that of the original image, the spatial resolution itself is the same as the original image. A blur correction such as a frequency enhancement process is performed for a quadruple-density sampling image acquired above in order to acquire a high-spatial resolution image. A known super-resolution technique can be used as the frequency enhancement process.

According to the present embodiment, a high-spatial resolution image can be acquired without changing a detector or the like of a conventional apparatus.

### Sixth Embodiment

The present embodiment is characterized by directly generating a CPR image using bed position information.

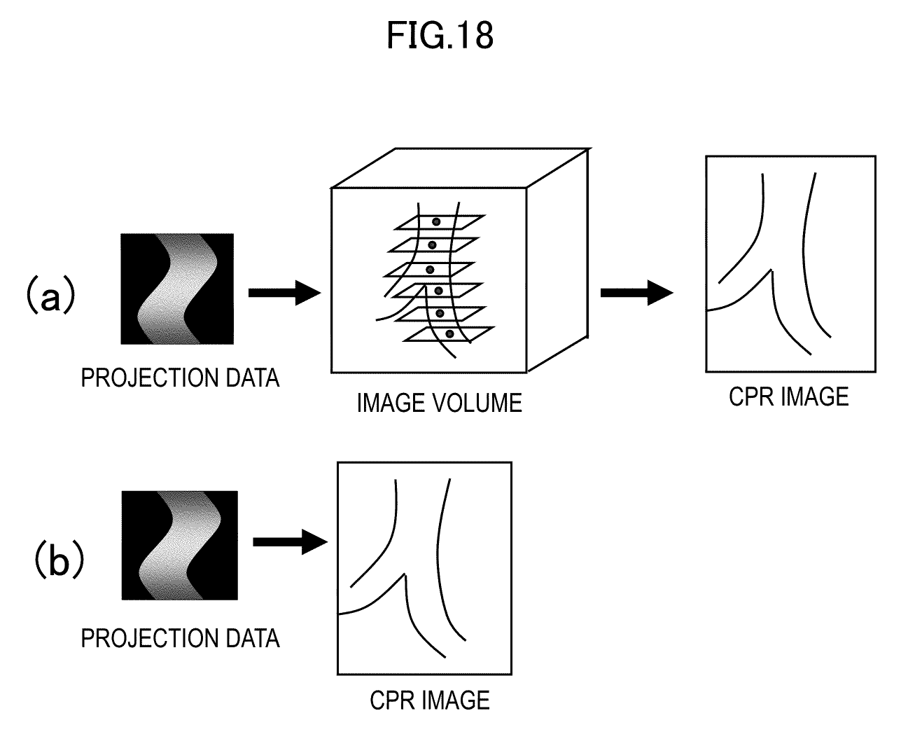

A conventional CPR image is an image along a lumen and is created by generating volume data (three-dimensional image data) from projection data of each slice and connecting the center points of the lumen in the volume data as shown in FIG. 18(a). That is, CPR image creation needs to generate volume data and images that are perpendicular to the line connecting the center points of the lumen and pass through the center points of the lumen by interpolation processing in the volume data. Therefore, the spatial resolution deteriorates. On the contrary to this, the present embodiment performs scanning while moving a bed along the line connecting the center points of a lumen as shown in FIG. 18(b) and can create a CPR image, not by interpolation in volume data but directly, using the bed movement information as the center point position information of the lumen for image reconstruction.

Hereinafter, operations of the X-ray CT apparatus of the present embodiment will be described. Also in the present embodiment, similarly to the first embodiment, a bed movement curve is set along the center points of a lumen based on a pre-scanned image, and scanning is performed while moving a bed according to the set bed movement curve (FIG. 5: S501 to S503). Next, a CPR image is reconstructed using the set bed position information or the bed position information measured during scanning.

A CPR surface can be regulated based on a coordinate of the center line and an angle (for which the body axis is set as the rotation axis), a CPR surface sημ coordinate and an XYZ coordinate can be associated with each other as the following formula when the reconstruction center coordinate for a μ_{I} coordinate of the CPR surface is set to (x₀(μ_{I}), y₀(μ_{I})).

[Formula 13]

x_{I}=s_{I} cos η_{I}+x₀(μ_{I}) (13-1)

y_{I}=s_{I} sin η_{I}+y₀(μ_{I}) (13-2)

z_{I}=ρ(μ_{I}) (13-3)

In the formula, “η_{I}” is an angle of a CPR surface (an angle for which the body axis is set as the rotation axis of the CPR surface), “s_{I}” is a position of a coordinate perpendicular to the z axis on the CPR surface, “μ_{I}” is a coordinate perpendicular to the s axis along the CPR surface, and “ρ” is a function for regulating travel on the CPR surface.

Based on the above relationship, the fan-beam-mode image reconstruction of the present embodiment for the CPR surface can be shown using the following formula (14).

$$\begin{matrix} [ Formula 14 ] & \\ I ( {s}_{I} , {η}_{I} , {μ}_{I} ) = \frac{1}{2 π} {\int }_{- π}^{π} \frac{{R}^{2}}{{L ( β , {x}_{I}^{′} , {y}_{I}^{′} )}^{2}} {fP}_{fan} ( β , {α}_{I} , {v}_{I} ) d β & ( 14 ) \\ wherein , & \\ L ( β , {x}_{I}^{′} , {y}_{I}^{′} ) = \sqrt{{( R - {x}_{I}^{′} \cdot sin β + {y}_{I}^{′} \cdot cos β )}^{2} + {( {x}_{I}^{′} \cdot cos β + {y}_{I}^{′} \cdot sin β )}^{2}} & ( 14 \text{-} 1 ) \\ {α}_{I} = arctan ( \frac{{x}_{I}^{′} \cdot cos β + {y}_{I}^{′} \cdot sin β}{R - {x}_{I}^{′} \cdot sin β + {y}_{I}^{′} \cdot cos β} ) & ( 14 \text{-} 2 ) \\ {v}_{I} = \frac{( ρ ( {μ}_{I} ) - {z}_{S}^{′} ) \cdot SID}{L ( β , {x}_{I}^{′} , {y}_{I}^{′} )} & ( 14 \text{-} 3 ) \\ {z}_{S}^{′} = \frac{cos γ ( β ) \cdot T ( β ) \cdot β}{2 π} + {z}_{S 0} & ( 14 \text{-} 4 ) \\ {x}_{I}^{′} = {s}_{I} cos {η}_{I} + {x}_{0} ( {μ}_{I} ) + {κ}_{x} ( β ) & ( 14 \text{-} 5 ) \\ {y}_{I}^{′} = {s}_{I} sin {η}_{I} + {y}_{0} ( {μ}_{I} ) + {κ}_{y} ( β ) + Δ {y}_{I} = {s}_{I} sin {η}_{I} + {y}_{0} ( {μ}_{I} ) + {κ}_{y} ( β ) + \frac{sin γ ( β ) \cdot T ( β ) \cdot β}{2 π} & ( 14 \text{-} 6 )\end{matrix}$$

Similarly, the parallel-beam-mode image reconstruction for the CPR image can be shown using the following formula (15).

$$\begin{matrix} [ Formula 15 ] & \\ I ( {s}_{I} , {η}_{I} , {μ}_{I} ) = \frac{1}{π} {\int }_{{B}_{s} ( {s}_{I} , {η}_{I} , {μ}_{I} )}^{{B}_{e} ( {s}_{I} , {η}_{I} , {μ}_{I} )} {fP}_{para} ( φ , {t}_{I}^{( n )} , {v}_{I} ) \cdot W ( φ - {B}_{s} ( {s}_{I} , {η}_{I} , {μ}_{I} ) - F π ) \cdot d φ & ( 15 ) \\ wherein , & \\ {v}_{I} ( {s}_{I} , {η}_{I} , {μ}_{I} , φ ) = \frac{( ρ ( {μ}_{I} ) - {z}_{S} ( {s}_{I} , {η}_{I} , {μ}_{I} , φ ) ) \cdot SID}{L ( {s}_{I} , {η}_{I} , {μ}_{I} , φ )} & ( 15 \text{-} 1 ) \\ {z}_{S} ( {s}_{I} , {η}_{I} , {μ}_{I} , φ ) = \frac{cos γ ( {β}^{( n )} ) \cdot T ( {β}^{( n )} ) \cdot ( φ + arc sin ( \frac{{t}_{I}^{( n )} ( {x}_{I}^{′} , {y}_{I}^{′} , φ )}{R} ) )}{2 π} + {z}_{S 0} & ( 15 \text{-} 2 ) \\ L ( {s}_{I} , {η}_{I} , {μ}_{I} , φ ) = D ( {s}_{I} , {η}_{I} , {μ}_{I} , φ ) + {w}_{I} ( {s}_{I} , {η}_{I} , {μ}_{I} , φ ) & ( 15 \text{-} 3 ) \\ D ( {s}_{I} , {η}_{I} , {x}_{0} , {y}_{0} , φ ) = \sqrt{{R}^{2} - {( {t}_{I}^{( n )} ( {s}_{I} , {η}_{I} , {x}_{0} , {y}_{0} , φ ) )}^{2}} & ( 15 \text{-} 4 ) \\ {w}_{I} ( {x}_{I}^{′} , {y}_{I}^{′} , φ ) = - {x}_{I}^{′} \cdot sin φ + ( {y}_{I}^{′} + Δ {y}_{I}^{( n - 1 )} ) \cdot cos φ & ( 15 \text{-} 5 ) \\ {t}_{I}^{( n )} ( {x}_{I}^{′} , {y}_{I}^{′} , φ ) = {x}_{I}^{′} \cdot cos φ + ( {y}_{I}^{′} + Δ {y}_{I}^{( n - 1 )} ) \cdot sin φ & ( 15 \text{-} 6 ) \\ Δ {y}_{I}^{( n )} ( {s}_{I} , {η}_{I} , {x}_{0} , {y}_{0} , φ ) = \frac{sin γ ( {β}^{( n )} ) \cdot T ( {β}^{( n )} ) \cdot ( φ + arcsin ( \frac{{t}_{I}^{( n - 1 )} ( {x}_{I}^{′} , {y}_{I}^{′} , φ )}{R} ) )}{2 π} & ( 15 \text{-} 7 ) \\ {β}^{( n )} = φ + α = φ + arctan \frac{{t}_{I}^{( n - 1 )}}{R} & ( 15 \text{-} 8 ) \\ {t}_{I}^{( 0 )} = ( {s}_{I} cos {η}_{I} + {x}_{0} ( {μ}_{I} ) ) \cdot cos φ + ( {s}_{I} sin {η}_{I} + {y}_{0} ( {μ}_{I} ) ) \cdot sin φ & ( 15 \text{-} 9 ) \\ {x}_{I}^{′} = {s}_{I} cos {η}_{I} + {x}_{0} ( {μ}_{I} ) + {κ}_{x} ( {β}^{( n )} ) & ( 15 \text{-} 10 ) \\ {y}_{I}^{′} = {s}_{I} sin {η}_{I} + {y}_{0} ( {μ}_{I} ) + {κ}_{y} ( {β}^{( n )} ) & ( 15 \text{-} 11 ) \\ n = 1 , 2 , 3 … & \end{matrix}$$

Although it is presumed that a bed position is moved in the vertical and horizontal directions during scanning in the image reconstruction using the above formula (14) or (15), the present embodiment can be applied also to a case of changing a scanner angle similarly to the second embodiment without moving the bed.

In this case, CPR image reconstruction using the above formula (14) or (15) can be performed by using scanner angle information of each slice as an angle of the CPR surface and setting a coordinate of the scanning center as the center coordinate of the CPR surface.

According to the present embodiment, there is no need to reconstruct volume data in advance, and a CPR image can be acquired directly by using bed position information to change a center point coordinate of a lumen for the CPR image reconstruction. Also, because the image was not reconstructed from the volume data, there is no image quality deterioration due to interpolation, which can acquire a CPR image with high spatial resolution.

Additionally, when a curve connecting the center points is obtained from a pre-scanned image even in case of not moving a bed in the vertical and horizontal directions during scanning and not changing a scanner tilt angle, a CPR image can be directly reconstructed by applying the image reconstruction of the present embodiment, and the present embodiment is a technique that can be applied also to an X-ray CT apparatus that is not provided with bed movement scanning. However, because the center point of a lumen is scanned so that it is always in the scanning center by performing the bed movement scanning, a CPR image describing spatial resolution of a lumen in the most favorable manner can be acquired.

### Seventh Embodiment

The present embodiment is characterized by adding a selection function for image reconstruction to an X-ray CT apparatus provided with a CPR image generating function by the above sixth embodiment and particularly features the selection GUI.

That is, the present embodiment also sets a change amount of a bed movement curve or a scanner tilt angle based on a pre-scanned image (S502) and performs scanning based on the bed movement curve or the tilt angle (S503) similarly to the sixth embodiment. The present embodiment has two types of image reconstruction modes for generating a CPR image (CPR mode) and generating a normal reconstruction image (volume mode) and is provided with a GUI for an operator to select a mode.

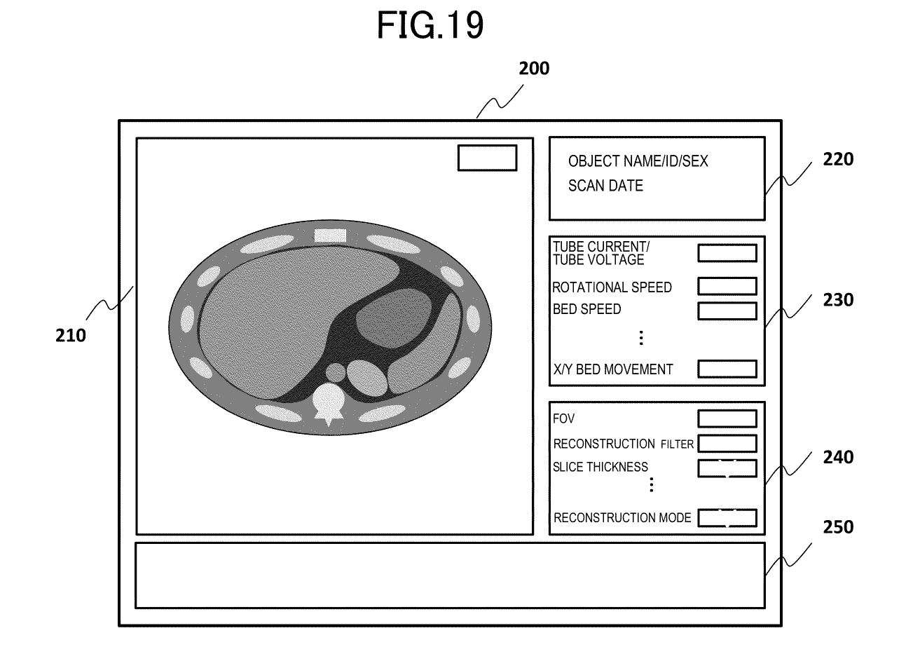

FIG. 19 shows a display window example of the present embodiment. The display window 200 shown in the diagram is comprised of the image display part 210, the object information display part 220, the scanning condition display part 230, the reconstruction condition display part 240, the status display part 250, and the like. The image display part 210 displays, for example, a positioning image of an object scanned previously, a CT image after image reconstruction, and a CPR image. The object information display part 220 displays information about an object such as an object name, sex, birth date, ID, and the like. The scanning condition display part 230 and the reconstruction condition display part 240 work as a GUI prompting an operator to input to the input unit 36 and displaying the input contents, and scanning conditions (a tube current, a tube voltage, a rotational speed, and a bed movement speed in the body-axis direction) and reconstruction conditions (a reconstruction mode, an image FOV, a reconstruction filter, an image slice thickness, and a reconstruction slice position) and reconstruction conditions are respectively set through the GUI. The status display part 250 displays a current status of the X-ray CT apparatus (scanning or idling) and a selected mode.

The scanning condition display part 230 displays a GUI to select scanning while a bed is moving vertically and horizontally or scanning by changing a scanner tilt angle as scanning conditions in addition to the above. Such a GUI may be displayed in a state where a positioning image is being displayed on the image display part 210 after the positioning image was scanned, and it may be configured so that scanning while a bed is moving vertically and horizontally and/or scanning by changing a scanner tilt angle are selectable at the same time. Additionally, when scanning while a bed is moving vertically and horizontally and/or scanning by changing a scanner tilt angle is selected, an operator can set a bed movement curve interactively, and the apparatus can determine a bed movement curve from the positioning image as described in the first embodiment.

Also, when scanning while a bed is moving vertically and horizontally and/or scanning by changing a scanner tilt angle is selected as scanning conditions, the reconstruction condition display part 240 displays a GUI to select the volume mode or the CPR mode as a reconstruction mode. The GUI may be a CPR selection button for selecting the CPR when the button is pressed or may be a check box or the like. When the CPR mode is selected as a reconstruction mode, a GUI for selecting the standard mode or the linear mode is displayed. Although a CPR image is an image along a vessel such as a blood vessel, the standard mode is a display mode for displaying a traveling direction of a blood vessel or the like as it is, and the linear mode is a display mode for setting so that a traveling direction of a blood vessel or the like corresponds to the Y direction (vertical direction) of the window.

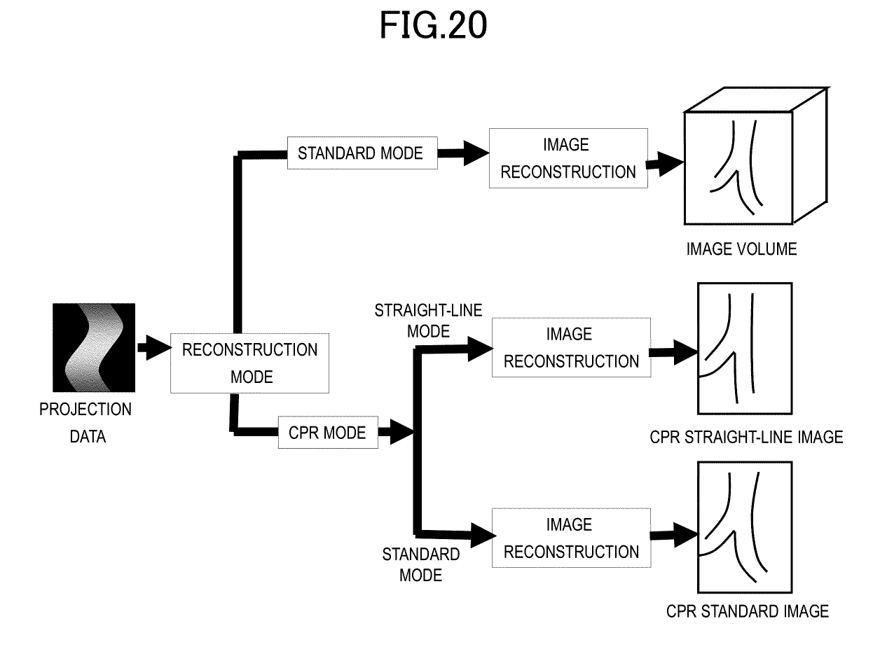

FIG. 20 shows a process flow when an operator operates the reconstruction condition display part 240 of such a display window. In a case where the CPR mode is selected as shown in the diagram, the image reconstruction described in the sixth embodiment is performed, and a CPR image (a linear image or standard image) is directly formed without reconstructing a volume image and is displayed on the image display part 210. In a case where the volume mode is selected, the image reconstruction described in the first and second embodiments is performed in order to generate volume image data. The volume image data is displayed on the image display part 210 after performing image processing such as known volume rendering. Also, a CPR image can be generated from the volume image data.

According to the present embodiment, scanning while a bed is moving vertically and horizontally and/or scanning by changing a scanner tilt angle can be performed smoothly, and the scanning-related conditions can be set smoothly.

### INDUSTRIAL APPLICABILITY

The present invention can provide a method for scanning while moving a bed vertically and horizontally or scanning by changing a scanner tilt angle and a new image reconstruction method supporting the above scanning method. Hence, a high spatial resolution image effective for diagnosis can be displayed.

### DESCRIPTION OF REFERENCE NUMERALS

10: scanner
11: X-ray generation device
12: X-ray detector
20: bed
23: bed movement measuring device
25: mechanical unit
30: computing device
31: bed movement amount setting unit
40: central controller
43: bed controller
100: X-ray CT apparatus
300: operation unit

1. A radiation tomographic apparatus comprising:
a bed that can place an object and move in the body-axis direction of the object;
a rotary disk that disposes a radiation source irradiating a radiation and a radiation detector oppositely across the bed and rotates around the bed;
an image generation unit that reconstructs a tomographic image of the object based on radiation data detected by the radiation detector while the rotary disk is rotating;
a mechanical unit that changes a position of the bed and/or an angle to the vertical plane of the rotary disk; a control unit that controls the mechanical unit; and a movement amount setting unit that sets a movement amount of the bed in a direction orthogonal to the body-axis direction and/or an angle change amount of the rotary disk during scanning,
wherein the control unit drives the mechanical unit to perform scanning according to the bed movement amount and/or the angle change amount of the rotary disk set by the movement amount setting unit, and the image generation unit generates an image using movement information of the bed in a direction orthogonal to the body-axis direction and/or angle information of the rotary disk during scanning.

2. The radiation tomographic apparatus according to claim 1,
wherein the control unit controls movement in a direction orthogonal to the body-axis direction of the bed in conjunction with the body-axis direction of the bed.

3. The radiation tomographic apparatus according to claim 2,
wherein the control unit is provided with a limit value calculation unit that calculates a limit value of a movement amount in a direction orthogonal to the body-axis direction of the bed for each position in the body-axis direction of the bed.

4. The radiation tomographic apparatus according to claim 3, including:
a display unit that displays an image generated by the image generation unit,
wherein the control unit allows the display unit to display the limit value of the movement amount calculated by the limit value calculation unit.

5. The radiation tomographic apparatus according to claim 1,
wherein the image generation unit generates an image using a movement amount in a direction orthogonal to the body-axis direction of the bed and/or an angle change amount of the rotary disk set by the movement amount setting unit.

6. The radiation tomographic apparatus according to claim 1,
wherein the mechanical unit is provided with a measuring unit that measures a movement amount in a direction orthogonal to the body-axis direction of the bed and/or an angle change amount of the rotary disk during scanning, and the image generation unit generates an image using the movement amount of the bed and/or the angle change amount of the rotary disk recorded by the measuring unit.

7. The radiation tomographic apparatus according to claim 1,
wherein the movement amount setting unit sets the movement amount of the bed in a direction orthogonal to the body-axis direction and/or the angle change amount of the rotary disk based on a pre-scanned image data of the object.

8. The radiation tomographic apparatus according to claim 7,
wherein the movement amount setting unit sets the movement amount of the bed so as to locate a site of interest of the object in the rotation center of the rotary disk.

9. The radiation tomographic apparatus according to claim 7,
wherein the movement amount setting unit sets the angle change amount of the rotary disk so that an angle of the rotary disk follows an inclination change to the body-axis direction at a site of interest of the object.

10. The radiation tomographic apparatus according to claim 1,
wherein the movement amount setting unit sets the movement amount in a direction orthogonal to the body-axis direction of the bed so as to change the movement amount linearly to a scanning view angle.

11. The radiation tomographic apparatus according to claim 10,
wherein the movement amount setting unit calculates the movement amount in a direction orthogonal to the body-axis direction of the bed based on a difference between a size of the radiation detector and an area of the object to be scanned.

12. The radiation tomographic apparatus according to claim 1,
wherein the control unit performs scanning whose number of views is greater than that required for one tomographic image for the same slice position and moves the bed in a direction orthogonal to the body-axis direction of the object at a pitch smaller than an element size of the radiation detector for the same slice position.

13. The radiation tomographic apparatus according to claim 1,
wherein the image generation unit generates a plurality of images whose positions are different in a direction orthogonal to the body-axis direction of the bed in order to generate a synthesized image by synthesizing the said plurality of the images.

14. The radiation tomographic apparatus according to claim 13,
wherein the image generation unit performs a frequency enhancement process for the synthesized image.

15. The radiation tomographic apparatus according to claim 1,
wherein the image generation unit generates a CPR image along a site of interest of the object using projection data scanned by changing a position in a direction orthogonal to the body-axis direction of the bed and/or an angle to the perpendicular surface of the rotary disk while moving the bed in the body-axis direction of the object.

16. The radiation tomographic apparatus according to claim 15, including:
an input device that allows an operator to select either of a three-dimensional image or a CPR image as an image to be generated by the image generation unit.

17. A radiation tomographic apparatus comprising:
a bed that places an object and can move in the body-axis direction of the object;
a rotary disk that disposes a radiation source irradiating a radiation and a radiation detector oppositely across the bed and rotates around the bed;
an image generation unit that reconstructs a tomographic image of the object based on radiation data detected by the radiation detector while the rotary disk is rotating;
and a recording unit that records coordinate variation along the body-axis direction in a site of interest of the object,
wherein the image generation unit generates a CPR image along the site of interest of the object using coordinate variation in the site of interest of the object recorded by the recording unit and projection data collected along the body-axis direction of the object.
